(19) United States
(12) Patent Application Publication
(10) Pub. No.: US 2005/0059654 A1
(43) Pub. Date: Mar. 17, 2005

(54) Method for treatment of depression and anxiety disorders by combination therapy

(76) Inventors: Stephen P. Arneric, Milan, MI (US); Cathryn M. Clary, New York, NY (US); Douglas Feltner, Novi, MI (US); Wilma M. Harrison, Harrison, NY (US); Richard J. Kavoussi, Ann Arbor, MI (US); Atul C. Pande, East Lyme, CT (US); Charles Price Taylor JR., Chelsea, MI (US)

Correspondence Address: WARNER-LAMBERT COMPANY, 2800 PLYMOUTH RD, ANN ARBOR, MI, 48105, US

(21) Appl. No.: 10/935,826

(22) Filed: Sep. 8, 2004

Related U.S. Application Data
(60) Provisional application No. 60/502,304, filed on Sep. 12, 2003.

(51) Int. Cl. A61K 31/519; A61K 31/195

(52) U.S. Cl. 514/220; 514/259.41; 514/561

(57) ABSTRACT

A method is provided of treating depression in mammals, including a human, as well as depression and a concomitant disease, disorder or condition exemplified by, but not limited to, anxiety, sleep disorder and post-traumatic stress disorder. The method comprises administering to the mammal in effective amount a combination of active ingredients comprising a) an alpha-2delta (A2D) ligand or a prodrug thereof, or a pharmaceutically acceptable salt of said A2D ligand or said prodrug and, active agents selected from; (b) a selective serotonin re-uptake inhibitor (SSRI) or a prodrug thereof or a pharmaceutically acceptable salt of said SSRI or said prodrug, (c) a selective noradrenaline re-uptake inhibitor (SNRI) or a prodrug thereof or a pharmaceutically acceptable salt of said SNRI or said prodrug and mixtures of (b) and (c). A pharmaceutical composition comprising a therapeutically effective amount (a) an A2D ligand or a prodrug thereof, or a pharmaceutically acceptable salt of said A2D ligand or said prodrug and active agents selected from; (b) an SSRI or a prodrug thereof or a pharmaceutically acceptable salt of said SSRI or said prodrug, (c) an SNRI or a prodrug thereof or a pharmaceutically acceptable salt of said SNRI or said prodrug and mixtures of (b) and (c) is also provided. Preferred active ingredients for the treatment and the pharmaceutical composition include pregabalin, gabapentin, sertraline and reboxetine.

This application is a United States utility application, which claims the benefit of priority to U.S. Provisional Application No. 60/502,304, filed Sep. 12, 2003.

### FIELD OF THE INVENTION

The present invention relates to a method of treating a subject, including a mammal, and particularly a human, suffering from depression or anxiety, depression with concomitant anxiety, attention deficit hyperactivity disorder (ADHD) with concomitant anxiety, as well as other diseases, disorders and conditions. The method comprises administering to the subject therapeutically effective amounts of (a) an alpha-2-delta (A2D) ligand and (b) a serotonin re-uptake inhibitor (SSRI) or (c) a noradrenaline re-uptake inhibitor (SNRI) or a combination of (a), (b) and (c). The present invention also relates to pharmaceutical compositions comprising an A2D ligand, an SSRI or an SNRI and optionally a pharmaceutically acceptable carrier, as well as pharmaceutical compositions comprising a combination of an A2D ligand, an SSRI and an SNRI and optionally a pharmaceutically acceptable carrier.

### BACKGROUND OF THE INVENTION

A2D ligands are agents whose major mode of action is binding at the A2D binding site on the voltage gated calcium channel. As is known in the art, calcium channels which are present in various tissues have a central role in regulating intracellular calcium ion concentrations, and are implicated in several vital processes in animals such as neurotransmitter release, muscle contraction, pacemaker activity and secretion of hormones and other substances. Changes in calcium influx into cells which are mediated through calcium channels have been implicated in various human diseases such as disorders of the central nervous system and cardiovascular disease. For example, changes to calcium influx into neuronal cells may be implicated in conditions such as epilepsy, stroke, brain trauma, Alzheimer's disease, multiinfarct dementia, other classes of dementia, Korsakoff's disease, neuropathy caused by a viral infection of the brain or spinal cord (e.g., human immunodeficiency viruses, etc.), amyotrophic lateral sclerosis, convulsions, seizures, Huntington's disease, amnesia, or damage to the nervous system resulting from reduced oxygen supply, poison or other toxic substances (See e.g., Goldin et al., U.S. Pat. No. 5,312,928). Additionally, changes to calcium influx into cardiovascular cells may be implicated in conditions such as cardiac arrhythmia, angina pectoris, hypoxic damage to the cardiovascular system, ischemic damage to the cardiovascular system, myocardial infarction, and congestive heart failure (Goldin et al., supra). Other pathological conditions associated with elevated intracellular free calcium levels include muscular dystrophy and hypertension (Steinhardt et al., U.S. Pat. No. 5,559,004).

A2D ligands have been described for a number of indications. The best known A2D ligand, gabapentin (NEURONTIN®), 1-(aminomethyl)-cyclohexylacetic acid, was first described in the patent literature in the patent family comprising U.S. Pat. No. 4,024,175. The compound is approved for the treatment of epilepsy and neuropathic pain.

A second A2D ligand, pregabalin, (S)-(+)-4-amino-3-(2-methylpropyl)butanoic acid, is described in European patent application publication number EP641330 as an anti-convulsant treatment useful in the treatment of epilepsy and in EP0934061 for the treatment of pain.

Gabapentin and pregabalin are specific examples of agents that have been shown to bind at the A2D site. Their interaction at the A2D site is associated with the reduction of neurotransmitter release from stimulated neuronal tissues. Both of these drugs are well tolerated anticonvulsive agents that have also been disclosed to be useful as anxiolytics (see for example, D. J.Wustrow, “Case History of Gabapentin and Pregabalin”, in program material “The 15^{th} Residential School on Medicinal Chemistry” held at Drew University, Madison, N.J., Jun. 11-15, 2001).

Many types of depression, mental, behavioral, and neurological disorders originate from disturbances in brain circuits that convey signals using certain monoamine neurotransmitters. Monoamine neurotransmitters include, for example, serotonin (5-HT), norepinephrine (noradrenaline), and dopamine.

These neurotransmitters travel from the terminal of a neuron across a small gap (i.e., the synaptic cleft) and bind to receptor molecules on the surface of a second neuron. This binding elicits intracellular changes that initiate or activate a response or change in the postsynaptic neuron. Inactivation occurs primarily by transport (i.e., reuptake) of the neurotransmitter back into the presynaptic neuron.

Selective serotonin reuptake inhibitors (SSRI's) function by inhibiting the reuptake of serotonin by afferent neurons. SSRI's well known in the art include, but are not limited to sertraline (Zoloft®), sertraline metabolite demethylsertraline, fluoxetine (Prozac®), norfluoxetine (fluoxetine desmethyl metabolite), fluvoxamine (Luvox®), paroxetine (Seroxat®, Paxil®) and its alternative formulation, Paxil-CR®, citalopram (Celexa®), citalopram metabolite desmethylcitalopram, escitalopram (Lexapro®), d,l-fenfluramine (Pondimin®), femoxetine, ifoxetine, cyanodothiepin, litoxetine, cericlamine, dapoxetine, nefazaodone (Serxone®), and trazodone (Desyrel®).

Selective noradrenaline or norepinephrine uptake inhibitors (SNRI's) relieve depression by increasing noradrenaline levels. SNRI's well known in the art include, but are not limited to, reboxetine (Edronax®) and all enantiomers of reboxetine, ie., (R/R,S/S,R/S,S/R), desipramine (Norpramin®), maprotiline (Ludiomil®), lofepramine (Gamanil®), mirtazepine (Remeron®), oxaprotiline, fezolamine, atomoxetine and buproprion (Wellbutrin®), buproprion metabolite hydroxybuproprion, nomifensine (Merital®), viloxazine (Vivalan®), or mianserin (Bolvidon®).

Pharmaceutical agents which inhibit the reuptake of both serotonin and norepinephrine include venlafaxine (Effexor®), venlafaxine metabolite O-desmethylvenlafaxine, clomipramine (Anafranil®), clomipramine metabolite desmethylclomipramine, duloxetine (Cymbalta®), milnacipran, and imipramine (Tofranil® or Janimine®).

Howard, in European patent application EP 1 254 668 A2, discloses another method to treat depression and anxiety that utilizes novel biaryl ether derivatives exhibiting serotonin reuptake inhibitor activity in combination with a GABA-A alpha 2/3 agonist.

U.S. Pat. No. 4,536,518 discloses certain cis-isomeric derivatives of 4-phenyl-1,2,3,4-tetrahydro-1-naphthalenamine, including sertraline, that are useful as antidepressants and that function by blocking the reuptake of serotonin. U.S. Pat. No. 6,197,819 refers to novel gamma amino butyric acid analogs including pregabalin useful for treating various central nervous system disorders such as epilepsy, Huntington's chorea, cerebral ischemia, and Parkinson's disease, as well as depression, anxiety and psychosis.

U.S. Pat. No. 4,024,175 refers to certain cyclic gamma-amino acid derivatives including gabapentin that are useful for treating various cerebral diseases such as epilepsy, faintness, hypokinesia and cranial traumas. U.S. Pat. No. 4,229,449 discloses (RS)-2-[(RS-alpha (2-ethoxyphenoxy)benzyl]-morpholine and its pharmaceutically acceptable salts (reboxetine); methods of preparation are described in U.S. Pat. No. 5,068,433 and in U.S. Pat. No. 5,391,735.

The contents of all patents and publications cited within the present application are hereby incorporated by reference.

### SUMMARY OF THE INVENTION

The present invention is directed to a method of treating a subject, including a mammal, and particularly a human, suffering from depression or anxiety with one or more concomitant condition, disease or disorder, or from post traumatic stress disorder, comprising administering to the subject a therapeutically effective amount of (a) an A2D ligand and (b) a serotonin re-uptake inhibitor (SSRI) or (c) a noradrenaline re-uptake inhibitor (SNRI) or a combination of (a), (b) and (c). In said method, (a) and, (b) or (c) or (a) and (b) and (c) may be administered in either a sequential or concurrent manner. In said method, (b) and (c) may be the same active agent. Said condition; disease or disorder concomitant with depression includes, but is not limited to, anxiety and sleep disorders including insomnia, alone or in combination.

The present invention is also directed to a method of treating a subject, including a mammal, and particularly a human, suffering from depression with therapeutically effective amounts of (a) an A2D ligand and, (b) a selective serotonin re-uptake inhibitor (SSRI) or (c) a selective noradrenaline (norepinephrine) re-uptake inhibitor (SNRI), or a combination thereof. In said method, (a), and (b) or (c) may be administered in either a sequential or concurrent manner. In said method, (b) and (c) may be the same active agent.

In another aspect, the present invention is directed to a method of treating a subject, including a mammal, and particularly a human, with therapeutically effective amounts of (a) an A2D ligand, and (b) a selective serotonin re-uptake inhibitor (SSRI) or (c) a selective noradrenaline re-uptake inhibitor (SNRI), or a combination thereof, wherein (a), and (b) or (c) may be administered in either a sequential or concurrent manner or wherein (b) and (c) may be the same active agent, said subject suffering from one or more condition, disease or disorder selected from: generalized anxiety disorder, major depressive disorder, dysthymia, premenstrual dysphoric disorder, depression with concomitant anxiety, post traumatic stress disorder, panic disorder, specific phobias, obsessive compulsive disorder (OCD), borderline personality disorder, sleep disorders including insomnia, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, deteriorated cerebral function in geriatric patients, chemical dependencies, premature ejaculation, premenstrual syndrome (PMS) associated mood and appetite disorder, hot flashes, cancer, post myocardial infarction, regulation of immune response, immune system disorders, prevention of stenosis, modification of feeding behavior, blocking carbohydrate cravings, late luteal phase dysphoric disorder, attention deficit hyperactivity disorder (ADHD) with or without comorbid anxiety, and tobacco withdrawal-associated symptoms.

In another aspect, the present invention is directed to a method of treating a subject, including a mammal, and particularly a human, with therapeutically effective amounts of (a) an A2D ligand and, (b) a selective serotonin re-uptake inhibitor (SSRI) or (c) a selective noradrenaline re-uptake inhibitor (SNRI), or a combination thereof, wherein (a) and, (b) or (c) may be administered in either a sequential or concurrent manner, or wherein (b) and (c) may be the same active agent, said subject suffering from one or more condition, disease or disorder selected from circadian rhythm disorders, psychoactive substance abuse and dependence, schizophrenia, paraphilias, sexual dysfunctions, stress related illnesses and personality disorders manifested by anger, rejection sensitivity, low mental or physical energy, circadian rhythm disorders, personality disorders including borderline and antisocial personality disorders, hyopochondriasis, late luteal phase dysphoric disorder, psychoactive substance use disorders, sexual disorders, and schizophrenia, and related symptoms including stress, worry, and lack of mental or physical energy.

In another aspect, the present invention is directed to a method of treating a subject, including a mammal, and particularly a human, with therapeutically effective amounts of (a) an A2D ligand, and (b) a selective serotonin re-uptake inhibitor (SSRI) or (c) a selective noradrenaline uptake inhibitor (SNRI), or a combination thereof, wherein (a) and, (b) or (c) may be administered in either a sequential or concurrent manner, or wherein (b) and (c) may be the same active agent, said subject suffering from one or more condition, disease or disorder selected from somatoform disorders, somatization disorder, conversion disorder, body dysmorphic disorder; glaucoma, or ocular hypertension, senile dementia and other forms of memory impairment, neurodegenerative diseases, amyotrophic lateral sclerosis, cerebellar dysfunction, glutamate neurotoxicity in pathophysiology of spinal cord injury induced by aortic cross-clamping, neurological lesions related to traumatic injuries, especially spinal, cranial or cranial-spinal injuries, mitochondrial diseases, including Kearns-Sayre syndrome, MERRF syndrome, MELAS syndrome and Leber's disease, and cerebrovascular disorders.

In another aspect, the present invention is directed to a method of treating a subject, including a mammal, and particularly a human, with therapeutically effective amounts of (a) an A2D ligand, and (b) a selective serotonin re-uptake inhibitor (SSRI) or (c) a selective noradrenaline re-uptake inhibitor (SNRI), or a combination thereof, wherein (a) and, (b) or (c) may be administered in either a sequential or concurrent manner, or wherein (b) and (c) may be the same active agent, said subject suffering from one or more condition, disease or disorder selected from neuro-AIDs including disorders involving dementia, cognitive disorders, myopathies, ocular disorders and all neurological symptoms associated with the HIV-1 virus, the cough that is observed in patients who are being maintained on an ACE inhibitor, benign positional vertigo, inflammatory diseases, physiological conditions associated with the use, or sequelae of use, of cocaine or other psychomotors stimulants, mania in all its various forms whether acute or chronic, single or recurrent, and bipolar disorder.

In another aspect, the present invention is directed to a method of treating a subject, including a mammal, and particularly a human, with therapeutically effective amounts of (a) an A2D ligand, and (b) a selective serotonin re-uptake inhibitor (SSRI) or (c) a selective noradrenaline re-uptake inhibitor (SNRI), or a combination thereof, wherein (a) and, (b) or (c) may be administered in either a sequential or concurrent manner, or wherein (b) and (c) may be the same active agent, said subject suffering from one or more condition, disease or disorder selected from phencyclidine (PCP) addiction, addiction to alcohol, cocaine addiction, nicotine addiction, , drug-induced, electroshock-induced, light-induced, amygdala-kindled, and audiogenic seizures, perinatal asphyxia, Alzheimer's disease, affective illness including cyclothymia to prevent episodes of cyclothymia, mania with exhibited irritability, distractibility, and poor judgment, bipolar depression, and persons predisposed to bipolar disorder to prevent episodes of bipolar disorder.

In another aspect, the present invention is directed to a method of treating a subject, including a mammal, and particularly a human, with therapeutically effective amounts of (a) an A2D ligand, and (b) a selective serotonin re-uptake inhibitor (SSRI) or (c) a selective noradrenaline re-uptake inhibitor (SNRI), or a combination thereof, wherein (a) and, (b) or (c) may be administered in either a sequential or concurrent manner, or wherein (b) and (c) may be the same active agent, said subject suffering from one or more condition, disease or disorder selected from effects of ethanol withdrawal syndrome including tremor, anxiety, attention deficit disorder (ADHD) with or without comorbid anxiety, convulsions, stroke, ischemia (in order to prevent neuronal damage), acute and chronic treatment of obesity, partial onset seizures, and primary generalized tonic-clonic seizures.

In another aspect, the present invention is directed to a method of treating a subject, including a mammal, and particularly a human, with therapeutically effective amounts of (a) an A2D ligand, and (b) a selective serotonin re-uptake inhibitor (SSRI) or (c) a selective noradrenaline re-uptake inhibitor (SNRI), or a combination thereof, wherein (a) and, (b) or (c) may be administered in either a sequential or concurrent manner, or wherein (b) and (c) may be the same active agent, said subject suffering from one or more condition, disease or disorder selected from anxiety disorders, such as panic disorder with or without agoraphobia, agoraphobia without history of panic disorder, animal and other phobias, social phobias including the generalized and non-generalized subtypes, obsessive-compulsive disorder, acute stress disorder, generalized or substance-induced anxiety disorder, neuroses, convulsions, and depressive or bipolar disorders, for example single-episode or recurrent major depressive disorder, dysthymic disorder, bipolar I and bipolar II manic disorders, and cyclothymic disorder.

In another aspect, the present invention is directed to a method of treating a subject, including a mammal, and particularly a human, with therapeutically effective amounts of (a) an A2D ligand, and (b) a selective serotonin re-uptake inhibitor (SSRI) or (c) a selective noradrenaline re-uptake inhibitor (SNRI), or a combination thereof, wherein (a) and (b) or (c) may be administered in either a sequential or concurrent manner, or wherein (b) and (c) may be the same active agent, said subject suffering from one or more condition, disease or disorder selected from cardiac disorders such as myocardial infarction, angina, stroke, pulmonary embolism, transient ischemic attack, deep vein thrombosis, thrombotic re-occlusion subsequent to a coronary intervention procedure (heart surgery or vascular surgery), peripheral vascular thrombosis, Syndrome X, heart failure, and a disorder in which a narrowing of at least one coronary artery occurs.

In another aspect, the present invention is directed to a method of treating a subject, including a mammal, and particularly a human, with therapeutically effective amounts of (a) an A2D ligand, and (b) a selective serotonin re-uptake inhibitor (SSRI) or (c) a selective noradrenaline re-uptake inhibitor (SNRI), or a combination thereof, wherein (a) and, (b) or (c) may be administered in either a sequential or concurrent manner, or wherein (b) and (c) may be the same active agent, said subject suffering from one or more condition, disease or disorder selected from sleep apneas, depression, seasonal affective disorders and dysthmia, avoidant personality disorder, social phobia; memory disorders including dementia, amnestic disorders and age-associated memory impairment; disorders of eating behavior, including anorexia nervosa and bulimia nervosa, obesity, neuroleptic-induced parkinsonism and tardive dyskinesias, endocrine disorders such as hyperprolactinaemia, vasospasm (particularly in the cerebral vasculature), and asthma.

In another aspect, the present invention is directed to a method of treating a subject, including a mammal, and particularly a human, with therapeutically effective amounts of (a) an A2D ligand, and (b) a selective serotonin re-uptake inhibitor (SSRI) or (c) a selective noradrenaline re-uptake inhibitor (SNRI), or a combination thereof, or a combination thereof, wherein (a) and, (b) or (c) may be administered in either a sequential or concurrent manner, or wherein (b) and (c) may be the same active agent, said subject suffering from one or more condition, disease or disorder selected from atherosclerosis, stuttering, chronic fatigue, alcohol abuse, appetite disorders, weight loss, agoraphobia, amnesia, smoking cessation, nicotine withdrawal syndrome symptoms, depressed mood and/or carbohydrate craving associated with pre-menstrual syndrome, disturbances of mood, disturbances of appetite or disturbances which contribute to recidivism associated with nicotine withdrawal, pre-menstrual dysphoric disorder, trichotillomania, symptoms following discontinuation of antidepressants, aggressive/intermittent explosive disorder, compulsive gambling, compulsive spending, compulsive sex, psychoactive substance use disorder, psychiatric symptoms such as worry, anger, rejection sensitivity, and lack of mental or physical energy, psychoactive substance abuse disorders and obsessive compulsive disorders, abuse of anabolic steroids and dementia of aging either alone or in any combination, or concomitant with depression. In still another aspect, the present inventor is directed to a method for treating a subject, including a mammal, with a therapeutically synergistic amount of (a) an A2D ligand, and (b) a selective serotonin re-uptake inhibitor (SSRI) or (c) a selective noradrenaline re-uptake inhibitor (SNRI), or a combination thereof, or wherein (a) and, (b) or (c) may be administered in either a sequential or concurrent manner, or wherein (b) and (c) may be the same active agent, said subject suffering from depression and/or anxiety.

In still another aspect, the present invention is directed to a pharmaceutical composition comprising a therapeutically effective amount of (a) an A2D ligand or a prodrug thereof, or a pharmaceutically acceptable salt of said A2D ligand or said prodrug, and, (b) a selective serotonin re-uptake inhibitor (SSRI) or a prodrug thereof or a pharmaceutically acceptable salt of said SSRI or said prodrug; or (c) a selective noradrenaline re-uptake inhibitor (SNRI) or a prodrug thereof or a pharmaceutically acceptable salt of said SNRI or said prodrug; or a combination of (a), (b) and (c) and, optionally, a pharmaceutically acceptable vehicle, carrier or diluent. In the pharmaceutical composition (b) and (c) may be the same active agent. The pharmaceutical composition may act in a synergistic manner.

A2D ligands preferred for the methods and pharmaceutical compositions of the present invention are gabapentin and pregabalin or any prodrug thereof or any pharmaceutically acceptable salt of said A2D ligand or said prodrug. Other A2D ligands known in the art may also be used in the methods and pharmaceutical compositions of the instant invention.

SSRI's useful for the methods and pharmaceutical compositions of the present invention include sertraline, fluoxetine, fluvoxamine, paroxetine, citalopram, escitalopram, d,l-fenfluramine, femoxetine, ifoxetine, cyanodothiepin, litoxetine, or any prodrug thereof or any pharmaceutically acceptable salt of said SSRI or said prodrug. Preferably, the SSRI is sertraline.

SNRI's useful for the methods and pharmaceutical compositions of the present invention include reboxetine, desipramine, maprotiline, lofepramine, mirtazepine, oxaprotiline, fezolamine, atomoxetine and buproprion or any prodrug thereof or any pharmaceutically acceptable salt of said SNRI or said prodrug. Preferably, the SNRI is reboxetine.

A preferred embodiment of the invention method utilizes an A2D ligand that is a cyclic amino acid compound of Formula I 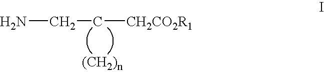
wherein R₁ is hydrogen or lower alkyl and n is an integer of from 4 to 6, and the pharmaceutically acceptable salts thereof. An especially preferred embodiment utilizes a compound of Formula I where R₁ is hydrogen and n is 5, which compound is 1-(aminomethyl)-cyclohexane acetic acid, known generically as gabapentin. Other preferred A2D ligands, or a pharmaceutically acceptable salt thereof, are compounds of Formula I wherein the cyclic ring is substituted, for example with alkyl such as methyl or ethyl. Typical of such compounds include (1-aminomethyl-3-methylcyclohexyl) acetic acid, (1-aminomethyl-3-methylcyclopentyl) acetic acid, and (1-aminomethyl-3,4-dimethylcyclopentyl) acetic acid.

In another preferred embodiment, the invention method utilizes an A2D ligand of Formula II 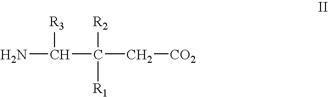
or a pharmaceutically acceptable salt thereof, wherein:
R₁ is a straight or branched unsubstituted alkyl of from 1 to 6 carbon atoms, unsubstituted phenyl, or unsubstituted cycloalkyl of from 3 to 6 carbon atoms;
R₂ is hydrogen or methyl; and
R₃ is hydrogen, methyl, or carboxyl.

Diastereomers and enantiomers of compounds of Formula II can be utilized in the invention method.

An especially preferred embodiment of the invention method employs a compound of Formula II where R₂ and R₃ are both hydrogen, and R₁ is —(CH₂)₀₋₂-i C₄H₉ as an (R), (S), or (R,S) isomer.

A more preferred embodiment of the invention method utilizes a compound of Formula II named 3-aminomethyl-5-methyl-hexanoic acid, or especially (S)-3-(aminomethyl)-5-methylhexanoic acid, now known generically as pregabalin. Pregabalin is also known as “CI-1008” and “S-(+)-3-IBG.”

Another preferred embodiment of the invention method utilizes a compound of Formula II named 3-(1-aminoethyl)-5-methylheptanoic acid or 3-(1-aminoethyl)-5-methylhexanoic acid.

Another preferred embodiment of the invention method utilizes an A2D ligand that is a compound of the Formula III, IIIC, IIIF, IIIG, or IIIH 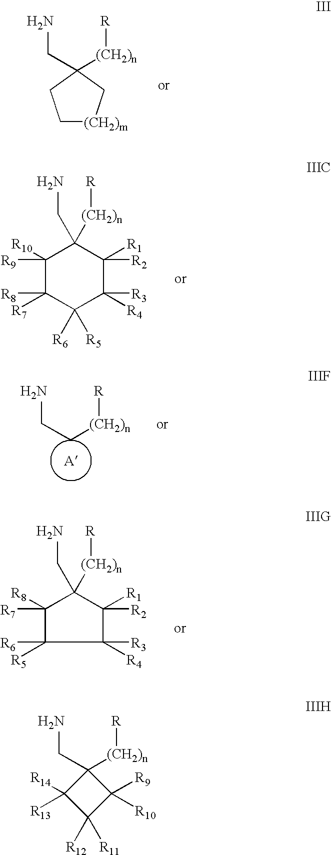
or a pharmaceutically acceptable salt thereof wherein:
n is an integer of from 0 to 2;
m is an integer of from 0 to 3;
R is sulfonamide,
   amide,
   phosphonic acid,
   heterocycle,
   sulfonic acid, or
   hydroxamic acid;
   with the proviso that R can not be sulfonic acid when m is 2 and n is 1;
R₁ to R₁₄ are each independently selected from hydrogen or straight or branched alkyl of from 1 to 6 carbons, unsubstituted or substituted benzyl or phenyl which substituents are selected from halogen, alkyl, alkoxy, hydroxy, carboxy, carboalkoxy, trifluoromethyl, and nitro;
A′ is a bridged ring selected from 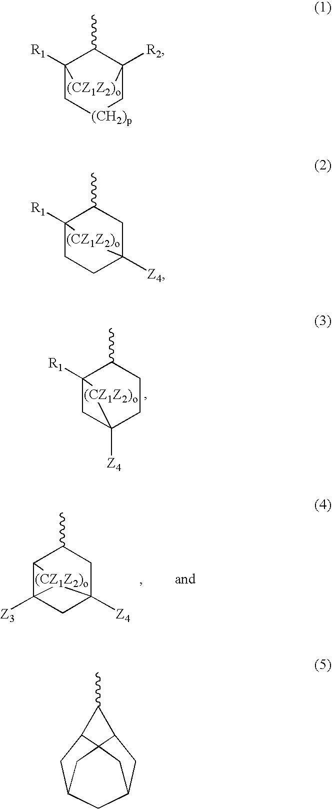
   wherein
[symbol] is the point of attachment;
Z₁ to Z₄ are each independently selected from hydrogen and methyl;
o is an integer of from 1 to 4; and
p is an integer of from 0 to 2.

Another preferred embodiment of the invention method utilizes a compound of Formulas III, IIIC, IIIF, IIIG, or IIIH selected from:
(1-Aminomethyl-cyclohexylmethyl)-phosphonic acid;
(1R-trans)(1-Aminomethyl-3-methyl-cyclohexylmethyl)-phosphonic acid;
(trans)(1-Aminomethyl-3,4-dimethyl-cyclopentylmethyl)-phosphonic acid;
(1R-trans)(1-Aminomethyl-3-methyl-cyclopentylmethyl)-phosphonic acid;
(1S-cis)(1-Aminomethyl-3-methyl-cyclopentylmethyl)-phosphonic acid;
(1S-trans)(1-Aminomethyl-3-methyl-cyclopentylmethyl)-phosphonic acid;
(1R-cis)(1-Aminomethyl-3-methyl-cyclopentylmethyl)-phosphonic acid;
(1α,3α,4α)(1-Aminomethyl-3,4-dimethyl-cyclopentylmethyl)-phosphonic acid;
(1α,3β,4β)(1-Aminomethyl-3,4-dimethyl-cyclopentylmethyl)-phosphonic acid;
(R)(1-Aminomethyl-3,3-dimethyl-cyclopentylmethyl)-phosphonic acid;
(S)(1-Aminomethyl-3,3-dimethyl-cyclopentylmethyl)-phosphonic acid;
(1-Aminomethyl-3,3-dimethyl-cyclobutylmethyl)-phosphonic acid;
2-(1-Aminomethyl-cyclohexyl)-N-hydroxy-acetamide;
(1S-trans)2-(1-Aminomethyl-3-methyl-cyclohexyl)-N-hydroxy-acetamide;
(trans)2-(1-Aminomethyl-3,4-dimethyl-cyclopentyl)-N-hydroxy-acetamide;
(1S-cis)2-(1-Aminomethyl-3-methyl-cyclopentyl)-N-hydroxy-acetamide;
(1R-trans)2-(1-Aminomethyl-3-methyl-cyclopentyl)-N-hydroxy-acetamide;
(1R-cis)2-(1-Aminomethyl-3-methyl-cyclopentyl)-N-hydroxy-acetamide;
(1S-trans)2-(1-Aminomethyl-3-methyl-cyclopentyl)-N-hydroxy-acetamide;
(1α,3α,4α)2-(1-Aminomethyl-3,4-dimethyl-cyclopentyl)-N-hydroxy-acetamide;
(1α,3β,4β)2-(1-Aminomethyl-3 ,4-dimethyl-cyclopentyl)-N-hydroxy-acetamide;
(S)2-(1-Aminomethyl-3,3-dimethyl-cyclopentyl)-N-hydroxy-acetamide;
(R)2-(1-Aminomethyl-3,3-dimethyl-cyclopentyl)-N-hydroxy-acetamide;
2-(1-Aminomethyl-3,3-dimethyl-cyclobutyl)-N-hydroxy-acetamide;
N-[2-(1-Aminomethyl-cyclohexyl)-ethyl]-methanesulfonamide;
(1S-cis)N-[2-(1-Aminomethyl-3-methyl-cyclohexyl)-ethyl]-methanesulfonamide;
(trans)N-[2-(1-Aminomethyl-3,4-dimethyl-cyclopentyl)-ethyl]-methanesulfonamide;
(1S-cis)N-[2-(1-Aminomethyl-3-methyl-cyclopentyl)-ethyl]-methanesulfonamide;
(1R-trans)N-[2-(1-Aminomethyl-3-methyl-cyclopentyl)-ethyl]-methanesulfonamide;
(1R-cis)N-[2-(1-Aminomethyl-3-methyl-cyclopentyl)-ethyl]-methanesulfonamide;
(1S-cis)N-[2-(1-Aminomethyl-3-methyl-cyclopentyl)-ethyl]-methanesulfonamide;
(1α,3α,4α)N-[2-(1-Aminomethyl-3,4-dimethyl-cyclopentyl)-ethyl]-methanesulfonamide;
(1α,3β,4β)N-[2-(1-Aminomethyl-3,4-dimethyl-cyclopentyl)-ethyl]-methanesulfonamide;
(S)N-[2-(1-Aminomethyl-3,3-dimethyl-cyclopentyl)-ethyl]-methanesulfonamide;
(R)N-[2-(1-Aminomethyl-3,3-dimethyl-cyclopentyl)-ethyl]-methanesulfonamide;
N-[2-(1-Aminomethyl-3,3-dimethyl-cyclobutyl)-ethyl]-methanesulfonamide;
(1S-cis)3-(1-Aminomethyl-3-methyl-cyclohexylmethyl)-4H-[1,2,4]oxadiazol-5-one;
(trans)3-(1-Aminomethyl-3,4-dimethyl-cyclopentylmethyl)-4H-[1,2,4]oxadiazol-5-one;
(1S-cis)3-(1-Aminomethyl-3-methyl-cyclopentylmethyl)-4H-[1,2,4]oxadiazol-5-one;
(1R-trans)3-(1-Aminomethyl-3-methyl-cyclopentylmethyl)-4H-[1,2,4]oxadiazol-5-one;
(1R-cis)3-(1-Aminomethyl-3-methyl-cyclopentylmethyl)-4H-[1,2,4]oxadiazol-5-one;
(1S-trans)3-(1-Aminomethyl-3-methyl-cyclopentylmethyl)-4H-[1,2,4]oxadiazol-5-one;
(1α,3α,4α)3-(1-Aminomethyl-3,4-dimethyl-cyclopentylmethyl)-4H-[1,2,4]oxadiazol-5-one;
(1α,3β,4β)3-(1-Aminomethyl-3,4-dimethyl-cyclopentylmethyl)-4H-[1,2,4]oxadiazol-5-one;
(S)3-(1-Aminomethyl-3,3-dimethyl-cyclopentylmethyl)-4H-[1,2,4]oxadiazol-5-one;
(R)3-(1-Aminomethyl-3,3-dimethyl-cyclopentylmethyl)-4H-[1,2,4]oxadiazol-5-one;
3-(1-Aminomethyl-3,3-dimethyl-cyclobutylmethyl)-4H-[1,2,4]oxadiazol-5-one;
3-(1-Aminomethyl-cyclohexylmethyl)-4H-[1,2,4]oxadiazole-5-thione;
(1S-cis)3-(1-Aminomethyl-3-methyl-cyclohexylmethyl)-4H-[1,2,4]oxadiazole-5-thione;
(trans)3-(1-Aminomethyl-3,4-dimethyl-cyclopentylmethyl)-4H-[1,2,4]oxadiazole-5-thione;
(1S-cis)3-(1-Aminomethyl-3-methyl-cyclopentylmethyl)-4H-[1,2,4]oxadiazole-5-thione;
(1R-trans)3-(1-Aminomethyl-3-methyl-cyclopentylmethyl)-4H-[1,2,4]oxadiazole-5-thione;
(1R-cis)3-(1-Aminomethyl-3-methyl-cyclopentylmethyl)-4H-[1,2,4]oxadiazole-5-thione;
(1S-trans)3-(1-Aminomethyl-3-methyl-cyclopentylmethyl)-4H-[1,2,4]oxadiazole-5-thione;
(1α,3α,4α)3-(1-Aminomethyl-3,4-dimethyl-cyclopentylmethyl)4H-[1,2,4]oxadiazole-5-thione;
(1α,3β,4β)3-(1-Aminomethyl-3,4-dimethyl-cyclopentylmethyl)-4H-[1,2,4]oxadiazole-5-thione;
(S)3-(1-Aminomethyl-3,3-dimethyl-cyclopentylmethyl)-4H-[1,2,4]oxadiazole-5-thione;
(R)3-(1-Aminomethyl-3,3-dimethyl-cyclopentylmethyl)-4H-[1,2,4]oxadiazole-5-thione;
3-(1-Aminomethyl-3,3-dimethyl-cyclobutylmethyl)-4H-[1,2,4]oxadiazole-5-thione;
C-[1-(1H-Tetrazol-5-ylmethyl)-cyclohexyl]-methylamine;
(1S-cis)C-[3-Methyl-1-(1H-tetrazol-5-ylmethyl)-cyclohexyl]-methylamine;
(trans)C-[3,4-Dimethyl-1-(1H-tetrazol-5-ylmethyl)-cyclopentyl]-methylamine;
(1S-cis)C-[3-Methyl-1-(1 H-tetrazol-5-ylmethyl)-cyclopentyl]-methylamine;
(1R-trans)C-[3-Methyl-1-(1H-tetrazol-5-ylmethyl)-cyclopentyl]-methylamine;
(1R-cis)C-[3-Methyl-1-(1H-tetrazol-5-ylmethyl)-cyclopentyl]-methylamine;
(1S-trans)C-[3-Methyl-1-(1H-tetrazol-5-ylmethyl)-cyclopentyl]-methylamine;
(1α,3α,4α)C-[3,4-Dimethyl-1-(1H-tetrazol-5-ylmethyl)-cyclopentyl]-methylamine;
(1α,3β,4β)C-[3,4-Dimethyl-1-(1H-tetrazol-5-ylmethyl)-cyclopentyl]-methylamine;
(S)C-[3,3-Dimethyl-1-(1H-tetrazol-5-ylmethyl)-cyclopentyl]-methylamine;
(R)C-[3,3-Dimethyl-1-(1H-tetrazol-5-ylmethyl)-cyclopentyl]-methylamine;
C-[3,3-Dimethyl-1-(1H-tetrazol-5-ylmethyl)-cyclobutyl]-methylamine;
N-[2-(1-Aminomethyl-cyclohexyl)-ethyl]-C,C,C-trifluoro-methanesulfonamide;
(1S-cis)N-[2-(1-Aminomethyl-3-methyl-cyclohexyl)-ethyl]-C,C,C-trifluoro-methanesulfonamide;
(trans)N-[2-(1-Aminomethyl-3,4-dimethyl-cyclopentyl)-ethyl]-C,C,C-trifluoro-methanesulfonamide;
(1R-cis)N-[2-(1-Aminomethyl-3-methyl-cyclopentyl)-ethyl]-C,C,C-trifluoro-methanesulfonamide;
(1S-trans)N-[2-(l -Aminomethyl-3-methyl-cyclopentyl)-ethyl]-C,C,C-trifluoro-methanesulfonamide;
(1S-cis)N-[2-(1-Aminomethyl-3-methyl-cyclopentyl)-ethyl]-C,C,C-trifluoro-methanesulfonamide;
(1R-trans)N-[2-(1-Aminomethyl-3-methyl-cyclopentyl)-ethyl]-C,C,C-trifluoro-methanesulfonamide;
(1α,3α,4α)N-[2-(1-Aminomethyl-3,4-dimethyl-cyclopentyl)-ethyl]-C,C,C-trifluoro-methanesulfonamide;
(1α,3β,4β)N-[2-(1-Aminomethyl-3,4-dimethyl-cyclopentyl)-ethyl]-C,C,C-trifluoro-methanesulfonamide;
(S)N-[2-(1-Aminomethyl-3,3-dimethyl-cyclopentyl)-ethyl]-C,C,C-trifluoro-methanesulfonamide;
(R)N-[2-(1-Aminomethyl-3,3-dimethyl-cyclopentyl)-ethyl]-C,C,C-trifluoro-methanesulfonamide;
N-[2-(1-Aminomethyl-3,3-dimethyl-cyclobutyl)-ethyl]-C,C,C-trifluoro-methanesulfonamide;
3-(1-Aminomethyl-cyclohexylmethyl)-4H-[1,2,4]thiadiazol-5-one;
(1S-cis)3-(1-Aminomethyl-3-methyl-cyclohexylmethyl)-4H-[1,2,4]thiadiazol-5-one;
(trans)3-(1-Aminomethyl-3,4-dimethyl-cyclopentylmethyl)-4H-[1,2,4]thiadiazol-5-one;
(1R-cis)3-(1-Aminomethyl-3-methyl-cyclopentylmethyl)-4H-[1,2,4]thiadiazol-5-one;
(1S-trans)3-(1-Aminomethyl-3-methyl-cyclopentylmethyl)-4H-[1,2,4]thiadiazol-5-one;
(1S-cis)3-(1-Aminomethyl-3-methyl-cyclopentylmethyl)-4H-[1,2,4]thiadiazol-5-one;
(1R-trans)3-(1-Aminomethyl-3-methyl-cyclopentylmethyl)-4H-[1,2,4]thiadiazol-5-one;
(1α,3α,4α)3-(1-Aminomethyl-3,4-dimethyl-cyclopentylmethyl)-4H-[1,2,4]thiadiazol-5-one;
(1α,3β,4β)3-(1-Aminomethyl-3,4-dimethyl-cyclopentylmethyl)-4H-[1,2,4]thiadiazol-5-one;
(S)3-(1-Aminomethyl-3,3-dimethyl-cyclopentylmethyl)-4H-[1,2,4]thiadiazol-5-one;
(R)3-(1-Aminomethyl-3,3-dimethyl-cyclopentylmethyl)-4H-[1,2,4]thiadiazol-5-one;
3-(1-Aminomethyl-3,3-dimethyl-cyclobutylmethyl)-4H-[1,2,4]thiadiazol-5-one;
C-[1-(2-Oxo-2,3-dihydro-2λ⁴-[1,2,3,5]oxathiadiazol-4-ylmethyl)-cyclohexyl]-methylamine;
(1S-cis)C-[3-Methyl-1-(2-oxo-2,3-dihydro-2λ⁴⁻[1,2,3,5]oxathiadiazol-4-ylmethyl)-cyclohexyl]-methyl amine;
(trans)C-[3,4-Dimethyl-1-(2-oxo-2,3-dihydro-2λ⁴⁻[1,2,3,5]oxathiadiazol-4-ylmethyl)-cyclopentyl]-methylamine;
(1S-cis)C-[3-Methyl-1-(2-oxo-2,3-dihydro-2λ⁴⁻[1,2,3,5]oxathiadiazol-4-ylmethyl)-cyclopentyl]-methylamine;
(1R-trans)C-[3-Methyl-1-(2-oxo-2,3-dihydro-2λ⁴⁻[1,2,3,5]oxathiadiazol-4-ylmethyl)-cyclopentyl]-methylamine;
(1R-cis)C-[3-Methyl-1-(2-oxo-2,3-dihydro-2λ⁴⁻[1,2,3,5]oxathiadiazol-4-ylmethyl)-cyclopentyl]-methylamine;
(1S-trans)C-[3-Methyl-1-(2-oxo-2,3-dihydro-2λ⁴⁻[1,2,3,5]oxathiadiazol-4-ylmethyl)-cyclopentyl]-methylamine;
(1α,3α,4α)C-[3,4-Dimethyl-1-(2-oxo-2,3-dihydro-2λ⁴⁻[1,2,3,5]oxathiadiazol-4-ylmethyl)-cyclopentyl]-methylamine;
(1α,3β,4β)C-[3,4-Dimethyl-1-(2-oxo-2,3-dihydro-2λ⁴⁻[1,2,3 ,5]oxathiadiazol-4-ylmethyl)-cyclopentyl]-methylamine;
(S)C-[3,3-Dimethyl-1-(2-oxo-2,3-dihydro-2λ⁴⁻[1,2,3,5]oxathiadiazol-4-ylmethyl)-cyclopentyl]-methylamine;
(R)C-[3,3-Dimethyl-1-(2-oxo-2,3-dihydro-2λ⁴⁻[1,2,3,5]oxathiadiazol-4-ylmethyl)-cyclopentyl]-methylamine;
C-[3,3-Dimethyl-1-(2-oxo-2,3-dihydro-2λ⁴⁻[1,2,3 ,5]oxathiadiazol-4-ylmethyl)-cyclobutyl]-methylamine;
(1-Aminomethyl-cyclohexyl)-methanesulfonamide;
(1R-trans)(1-Aminomethyl-3-methyl-cyclohexyl)-methanesulfonamide;
(trans)(1-Aminomethyl-3,4-dimethyl-cyclopentyl)-methanesulfonamide;
(1S-trans)(1-Aminomethyl-3-methyl-cyclopentyl)-methanesulfonamide;
(1R-cis)(1-Aminomethyl-3-methyl-cyclopentyl)-methanesulfonamide;
(1R-trans)(1-Aminomethyl-3-methyl-cyclopentyl)-methanesulfonamide;
(1S-cis)(1-Aminomethyl-3-methyl-cyclopentyl)-methanesulfonamide;
(1α,3β,4β)(1-Aminomethyl-3,4-dimethyl-cyclopentyl)-methanesulfonamide;
(1α,3α,4α)(1-Aminomethyl-3,4-dimethyl-cyclopentyl)-methanesulfonamide;
(R)(1-Aminomethyl-3,3-dimethyl-cyclopentyl)-methanesulfonamide;
(S)(1-Aminomethyl-3,3-dimethyl-cyclopentyl)-methanesulfonamide;
(1-Aminomethyl-3,3-dimethyl-cyclobutyl)-methanesulfonamide;
(1-Aminomethyl-cyclohexyl)-methanesulfonic acid;
(1R-trans) (1-Aminomethyl-3-methyl-cyclohexyl)-methanesulfonic acid;
(trans)(1-Aminomethyl-3,4-dimethyl-cyclopentyl)-methanesulfonic acid;
(1S-trans)(1-Aminomethyl-3-methyl-cyclopentyl)-methanesulfonic acid;
(1S-cis)(1-Aminomethyl-3-methyl-cyclopentyl)-methanesulfonic acid;
(1R-trans)(1-Aminomethyl-3-methyl-cyclopentyl)-methanesulfonic acid;
(1R-cis)(1-Aminomethyl-3-methyl-cyclopentyl)-methanesulfonic acid;
(1α,3β,4β)(1-Aminomethyl-3,4-dimethyl-cyclopentyl)-methanesulfonic acid;
(1α,3α,4α)(1-Aminomethyl-3,4-dimethyl-cyclopentyl)-methanesulfonic acid;
(R)(1-Aminomethyl-3,3-dimethyl-cyclopentyl)-methanesulfonic acid;
(S)(1-Aminomethyl-3,3-dimethyl-cyclopentyl)-methanesulfonic acid;
(1-Aminomethyl-3,3-dimethyl-cyclobutyl)-methanesulfonic acid;
(1-Aminomethyl-cyclopentylmethyl)-phosphonic acid;
2-(1-Aminomethyl-cyclopentyl)-N-hydroxy-acetamide;
N-[2-(1-Aminomethyl-cyclopentyl)-ethyl]-methanesulfonamide;
3-(1-Aminomethyl-cyclopentylmethyl)-4H-[1,2,4]oxadiazol-5-one;
3-(1-Aminomethyl-cyclopentylmethyl)-4H-[1,2,4]oxadiazole-5-thione;
C-[1-(1H-Tetrazol-5-ylmethyl)-cyclopentyl]-methylamine;
N-[2-(1-Aminomethyl-cyclopentyl)-ethyl]-C,C,C-trifluoro-methanesulfonamide;
3-(1-Aminomethyl-cyclopentylmethyl)-4H-[1,2,4]thiadiazol-5-one;
C-[1-(2-Oxo-2,3-dihydro-2λ⁴⁻[1,2,3,5]oxathiadiazol-4-ylmethyl)-cyclopentyl]-methyl amine;
(1-Aminomethyl-cyclopentyl)-methanesulfonamide;
(1-Aminomethyl-cyclopentyl)-methanesulfonic acid;
(9-Aminomethyl-bicyclo[3.3.1]non-9-ylmethyl)-phosphonic acid;
2-(9-Aminomethyl-bicyclo[3.3.1 ]non-9-yl)-N-hydroxy-acetamide;
N-[2-(9-Aminomethyl-bicyclo[3.3.1 ]non-9-yl)-ethyl]-methanesulfonamide;
3-(9-Aminomethyl-bicyclo[3.3.1 ]non-9-ylmethyl)-4H-[1,2,4]oxadiazol-5-one;
3-(9-Aminomethyl-bicyclo[3.3.1]non-9-ylmethyl)-4H-[1,2,4]oxadiazole-5-thione;
C-[9-(1H-Tetrazol-5-ylmethyl)-bicyclo[3.3.1]non-9-yl]-methylamine;
N-[2-(9-Aminomethyl-bicyclo[3.3.1]non-9-yl)-ethyl]-C,C,C-trifluoro-methanesulfonamide;
3-(9-Aminomethyl-bicyclo[3.3.1]non-9-ylmethyl)-4H-[1,2,4]thiadiazol-5-one;
C-[9-(2-Oxo-2,3-dihydro-2λ⁴⁻[1,2,3,5]oxathiadiazol-4-ylmethyl)-bicyclo[3.3.1]non-9-yl]-methylamine;
(9-Aminomethyl-bicyclo[3.3.1]non-9-yl)-methanesulfonamide;
(9-Aminomethyl-bicyclo[3.3.1]non-9-yl)-methanesulfonic acid;
(2-Aminomethyl-adamantan-2-ylmethyl)-phosphonic acid;
2-(2-Aminomethyl-adamantan-2-yl)-N-hydroxy-acetamide;
N-[2-(2-Aminomethyl-adamantan-2-yl)-ethyl]-methanesulfonamide;
3-(2-Aminomethyl-adamantan-2-ylmethyl)-4H-[1,2,4]oxadiazol-5-one;
3-(2-Aminomethyl-adamantan-2-ylmethyl)-4H-[1,2,4]oxadiazole-5-thione;
C-[2-(1H-Tetrazol-5-ylmethyl)-adamantan-2-yl]-methylamine;
N-[2-(2-Aminomethyl-adamantan-2-yl)-ethyl]-C,C,C-trifluoro-methanesulfonamide;
3-(2-Aminomethyl-adamantan-2-ylmethyl)-4H-[1,2,4]thiadiazol-5-one;
C-[2-(2-Oxo-2,3-dihydro-2λ⁴⁻[1,2,3,5]oxathiadiazol-4-ylmethyl)-adamantan-2-yl]-methyl amine;
(2-Aminomethyl-adamantan-2-yl)-methanesulfonamide;
(2-Aminomethyl-adamantan-2-yl)-methanesulfonic acid;
(1-Aminomethyl-cycloheptylmethyl)-phosphonic acid;
2-(1-Aminomethyl-cycloheptyl)-N-hydroxy-acetamide;
N-[2-(1-Aminomethyl-cycloheptyl)-ethyl]-methanesulfonamide;
3-(1-Aminomethyl-cycloheptylmethyl)-4H-[1,2,4]oxadiazole-5-thione;
N-[2-(1-Aminomethyl-cycloheptyl)-ethyl]-C,C,C-trifluoro-methanesulfonamide;
C-[1-(2-Oxo-2,3-dihydro-2λ⁴⁻[1,2,3,5]oxathiadiazol-4-ylmethyl)-cycloheptyl]-methylamine;
(1-Aminomethyl-cycloheptyl)-methanesulfonamide; and
(1-Aminomethyl-cycloheptyl)-methanesulfonic acid.

Another preferred embodiment of the invention method utilizes a compound of the Formula III, IIIC, IIIF, IIIG, or IIIH, wherein preferred compounds are those wherein R is a sulfonamide selected from —NHSO₂R¹⁵ or —SO₂NHR¹⁵ wherein R¹⁵ is straight or branched alkyl or trifluoromethyl.

Another preferred embodiment of the invention method utilizes a compound of the Formula m, IIIC, IIIF, IIIG, or IIIH, wherein especially preferred is N-[2-(1-aminomethyl-cyclohexyl)-ethyl]-methanesulfonamide.

Another preferred embodiment of the invention method utilizes a compound of the Formula III, IIIC, IIIF, IIIG, or IIIH, wherein other preferred compounds are those wherein R is a phosphonic acid, —PO₃H₂.

Another preferred embodiment of the invention method utilizes a compound of the Formula III, IIIC, IIIF, IIIG, or IIIH, wherein especially preferred are (1-aminomethyl-cyclohexylmethyl)-phosphonic acid and (2-aminomethyl-4-methyl-pentyl)-phosphonic acid.

Another preferred embodiment of the invention method utilizes a compound of the Formula III, IIIC, IIIF, IIIG, or IIIH, wherein other preferred compounds are those wherein R is a heterocycle selected from:

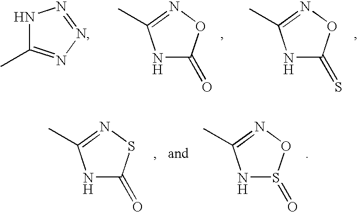

Another preferred embodiment of the invention method utilizes a compound of the Formula m, IIIC, IIIF, IIIG, or IIIH, wherein especially preferred are C-[1-(1H-tetrazol-5-ylmethyl)cyclohexyl]-methylamine and 4-methyl-2-(1H-tetrazol-5-ylmethyl)-pentylamine.

An especially preferred embodiment of the invention method utilizes a compound of the Formula III wherein:
m is an integer of from 0 to 2;
p is an integer of 2; and

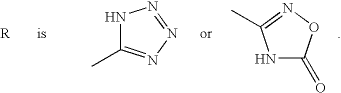

Still more preferred is an embodiment of the invention method which utilizes a compound of the Formula III, IIIC, IIIF, IIIG, or IIIH named 3-(1-aminomethyl-cyclohexylmethyl)-4H-[1,2,4]oxadiazol-5-one, or a pharmaceutically acceptable salt thereof.

Still more preferred is an embodiment of the invention method which utilizes a compound of the Formula III, IIIC, IIIF, IIIG, or IIIH named 3-(1-aminomethyl-cyclohexylmethyl)-4H-[1,2,4]oxadiazol-5-one hydrochloride.

Also preferred is an embodiment of the invention method which utilizes a compound of the Formula III, IIIC, IIIF, IIIG, or IIIH named 3-(1-aminomethyl-cycloheptylmethyl)-4H-[1,2,4]oxadiazol-5-one, or a pharmaceutically acceptable salt thereof.

Also more preferred is an embodiment of the invention method which utilizes a compound of the Formula III, IIIC, IIIF, IIIG, or IIIH named 3-(1-aminomethyl-cycloheptylmethyl)-4H-[1,2,4]oxadiazol-5-one hydrochloride.

Also preferred is an embodiment of the invention method which utilizes a compound of the Formula III, IIIC, IIIF, IIIG, or IIIH named C-[1-(1H-tetrazol-5-ylmethyl)-cycloheptyl]-methylamine, or a pharmaceutically acceptable salt thereof.

Also more preferred is an embodiment of the invention method which utilizes a compound of the Formula III, IIIC, IIIF, IIIG, or IIIH named C-[1-(1H-tetrazol-5-ylmethyl)-cycloheptyl]-methylamine.

Another preferred embodiment of the invention method utilizes an A2D ligand that is a compound of the Formula IV 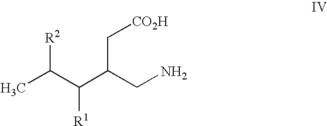
or a pharmaceutically acceptable salt thereof wherein:
R¹ is hydrogen, straight or branched alkyl of from 1 to 6 carbon atoms or phenyl;
R² is straight or branched alkyl of from 1 to 8 carbon atoms,
   straight or branched alkenyl of from 2 to 8 carbon atoms,
   cycloalkyl of from 3 to 7 carbon atoms,
   alkoxy of from 1 to 6 carbon atoms,
      -alkylcycloalkyl,
      -alkylalkoxy,
      -alkyl OH
      -alkylphenyl,
      -alkylphenoxy,
      -phenyl or substituted phenyl; and
R¹ is straight or branched alkyl of from 1 to 6 carbon atoms or phenyl when R² is methyl.

Preferred is an embodiment of the invention method employing a compound of Formula IV wherein R¹ is hydrogen, and R² is alkyl.

Another preferred embodiment of the invention method employing a compound of Formula IV wherein R¹ is methyl, and R² is alkyl.

Still another preferred embodiment of the invention method utilizes a compound of Formula IV wherein R¹ is methyl, and R² is methyl or ethyl.

Especially preferred is an embodiment of the invention method utilizing a compound of Formula IV selected from:
3-Aminomethyl-5-methylheptanoic acid;
3-Aminomethyl-5-methyl-octanoic acid;
3-Aminomethyl-5-methyl-nonanoic acid;
3-Aminomethyl-5-methyl-decanoic acid;
3-Aminomethyl-5-methyl-undecanoic acid;
3-Aminomethyl-5-methyl-dodecanoic acid;
3-Aminomethyl-5-methyl-tridecanoic acid;
3-Aminomethyl-5-cyclopropyl-hexanoic acid;
3-Aminomethyl-5-cyclobutyl-hexanoic acid;
3-Aminomethyl-5-cyclopentyl-hexanoic acid;
3-Aminomethyl-5-cyclohexyl-hexanoic acid;
3-Aminomethyl-5-trifluoromethyl-hexanoic acid;
3-Aminomethyl-5-phenyl-hexanoic acid;
3-Aminomethyl-5-(2-chlorophenyl)-hexanoic acid;
3-Aminomethyl-5-(3-chlorophenyl)-hexanoic acid;
3-Aminomethyl-5-(4-chlorophenyl)-hexanoic acid;
3-Aminomethyl-5-(2-methoxyphenyl)-hexanoic acid;
3-Aminomethyl-5-(3-methoxyphenyl)-hexanoic acid;
3-Aminomethyl-5-(4-methoxyphenyl)-hexanoic acid; and
3-Aminomethyl-5-(phenylmethyl)-hexanoic acid.

Another especially preferred embodiment of the invention method uses a compound of Formula IV selected from:
(3R,4S)-3-Aminomethyl-4,5-dimethyl-hexanoic acid;
3-Aminomethyl-4,5-dimethyl-hexanoic acid;
(3R,4S)-3-Aminomethyl-4,5-dimethyl-hexanoic acid MP;
(3S,4S)-3-Aminomethyl-4,5-dimethyl-hexanoic acid;
(3R,4R)-3-Aminomethyl-4,5-dimethyl-hexanoic acid MP;
3-Aminomethyl-4-isopropyl-hexanoic acid;
3-Aminomethyl-4-isopropyl-heptanoic acid;
3-Aminomethyl-4-isopropyl-octanoic acid;
3-Aminomethyl-4-isopropyl-nonanoic acid;
3-Aminomethyl-4-isopropyl-decanoic acid; and
3-Aminomethyl-4-phenyl-5-methyl-hexanoic acid.

Another preferred embodiment of the invention method uses a compound of Formula IV selected from:
(3S,5S)-3-Aminomethyl-5-methoxy-hexanoic acid;
(3S,5S)-3-Aminomethyl-5-ethoxy-hexanoic acid;
(3S,5S)-3-Aminomethyl-5-propoxy-hexanoic acid;
(3S,5S)-3-Aminomethyl-5-isopropoxy-hexanoic acid;
(3S,5S)-3-Aminomethyl-5-tert-butoxy-hexanoic acid;
(3S,5S)-3-Aminomethyl-5-fluoromethoxy-hexanoic acid;
(3S,5S)-3-Aminomethyl-5-(2-fluoro-ethoxy)-hexanoic acid;
(3S,5S)-3-Aminomethyl-5-(3,3,3-trifluoro-propoxy)-hexanoic acid;
(3S,5S)-3-Aminomethyl-5-phenoxy-hexanoic acid;
(3S,5S)-3-Aminomethyl-5-(4-chloro-phenoxy)-hexanoic acid;
(3S,5S)-3-Aminomethyl-5-(3-chloro-phenoxy)-hexanoic acid;
(3S,5S)-3-Aminomethyl-5-(2-chloro-phenoxy)-hexanoic acid;
(3S,5S)-3-Aminomethyl-5-(4-fluoro-phenoxy)-hexanoic acid;
(3S,5S)-3-Aminomethyl-5-(3-fluoro-phenoxy)-hexanoic acid;
(3S,5S)-3-Aminomethyl-5-(2-fluoro-phenoxy)-hexanoic acid;
(3S,5S)-3-Aminomethyl-5-(4-methoxy-phenoxy)-hexanoic acid;
(3S,5S)-3-Aminomethyl-5-(3-methoxy-phenoxy)-hexanoic acid;
(3S,5S)-3-Aminomethyl-5-(2-methoxy-phenoxy)-hexanoic acid;
(3S,5S)-3-Aminomethyl-5-(4-nitro-phenoxy)-hexanoic acid;
(3S,5S)-3-Aminomethyl-5-(3-nitro-phenoxy)-hexanoic acid;
(3S,5S)-3-Aminomethyl-5-(2-nitro-phenoxy)-hexanoic acid;
(3S,5S)-3-Aminomethyl-6-hydroxy-5-methyl-hexanoic acid;
(3S,5S)-3-Aminomethyl-6-methoxy-5-methyl-hexanoic acid;
(3S,5S)-3-Aminomethyl-6-ethoxy-5-methyl-hexanoic acid;
(3S,5S)-3-Aminomethyl-5-methyl-6-propoxy-hexanoic acid;
(3S,5S)-3-Aminomethyl-6-isopropoxy-5-methyl-hexanoic acid;
(3S,5S)-3-Aminomethyl-6-tert-butoxy-5-methyl-hexanoic acid;
(3S,5S)-3-Aminomethyl-6-fluoromethoxy-5-methyl-hexanoic acid;
(3S,5S)-3-Aminomethyl-6-(2-fluoro-ethoxy)-5-methyl-hexanoic acid;
(3S,5S)-3-Aminomethyl-5-methyl-6-(3,3,3-trifluoro-propoxy)-hexanoic acid;
(3S,5S)-3-Aminomethyl-5-methyl-6-phenoxy-hexanoic acid;
(3S,5S)-3-Aminomethyl-6-(4-chloro-phenoxy)-5-methyl-hexanoic acid;
(3S,5S)-3-Aminomethyl-6-(3-chloro-phenoxy)-5-methyl-hexanoic acid;
(3S,5S)-3-Aminomethyl-6-(2-chloro-phenoxy)-5-methyl-hexanoic acid;
(3S,5S)-3-Aminomethyl-6-(4-fluoro-phenoxy)-5-methyl-hexanoic acid;
(3S,5S)-3-Aminomethyl-6-(3-fluoro-phenoxy)-5-methyl-hexanoic acid;
(3S,5S)-3-Aminomethyl-6-(2-fluoro-phenoxy)-5-methyl-hexanoic acid;
(3S,5S)-3-Aminomethyl-6-(4-methoxy-phenoxy)-5-methyl-hexanoic acid;
(3S,5S)-3-Aminomethyl-6-(3-methoxy-phenoxy)-5-methyl-hexanoic acid;
(3S,5S)-3-Aminomethyl-6-(2-methoxy-phenoxy)-5-methyl-hexanoic acid;
(3S,5S)-3-Aminomethyl-5-methyl 6-(4-trifluoromethyl-phenoxy)-hexanoic acid;
(3S,5S)-3-Aminomethyl-5-methyl 6-(3-trifluoromethyl-phenoxy)-hexanoic acid;
(3S,5S)-3-Aminomethyl-5-methyl 6-(2-trifluoromethyl-phenoxy)-hexanoic acid;
(3S,5S)-3-Aminomethyl-5-methyl 6-(4-nitro-phenoxy)-hexanoic acid;
(3S,5S)-3-Aminomethyl-5-methyl 6-(3-nitro-phenoxy)-hexanoic acid;
(3S,5S)-3-Aminomethyl-5-methyl 6-(2-nitro-phenoxy)-hexanoic acid;
(3S,5S)-3-Aminomethyl-6-benzyloxy-5-methyl-hexanoic acid;
(3S,5S)-3-Aminomethyl-7-hydroxy-5-methyl-heptanoic acid;
(3S,5S)-3-Aminomethyl-7-methoxy-5-methyl-heptanoic acid;
(3S,5S)-3-Aminomethyl-7-ethoxy-5-methyl-heptanoic acid;
(3S,5S)-3-Aminomethyl-5-methyl-7-propoxy-heptanoic acid;
(3S,5S)-3-Aminomethyl-7-isopropoxy-5-methyl-heptanoic acid;
(3S,5S)-3-Aminomethyl-7-tert-butoxy-5-methyl-heptanoic acid;
(3S,5S)-3-Aminomethyl-7-fluoromethoxy-5-methyl-heptanoic acid;
(3S,5S)-3-Aminomethyl-7-(2-fluoro-ethoxy)-5-methyl-heptanoic acid;
(3S,5S)-3-Aminomethyl-5-methyl-7-(3,3,3-trifluoro-propoxy)-heptanoic acid;
(3S,5S)-3-Aminomethyl-7-benzyloxy-5-methyl-heptanoic acid;
(3S,5S)-3-Aminomethyl-5-methyl-7-phenoxy-heptanoic acid;
(3S,5S)-3-Aminomethyl-7-(4-chloro-phenoxy)-5-methyl-heptanoic acid;
(3S,5S)-3-Aminomethyl-7-(3-chloro-phenoxy)-5-methyl-heptanoic acid;
(3S,5S)-3-Aminomethyl-7-(2-chloro-phenoxy)-5-methyl-heptanoic acid;
(3S,5S)-3-Aminomethyl-7-(4-fluoro-phenoxy)-5-methyl-heptanoic acid;
(3S,5S)-3-Aminomethyl-7-(3-fluoro-phenoxy)-5-methyl-heptanoic acid;
(3S,5S)-3-Aminomethyl-7-(2-fluoro-phenoxy)-5-methyl-heptanoic acid;
(3S,5S)-3-Aminomethyl-7-(4-methoxy-phenoxy)-5-methyl-heptanoic acid;
(3S,5S)-3-Aminomethyl-7-(3- methoxy -phenoxy)-5-methyl-heptanoic acid;
(3S,5S)-3-Aminomethyl-7-(2- methoxy -phenoxy)-5-methyl-heptanoic acid;
(3S,5S)-3-Aminomethyl-5-methyl-7-(4-trifluoromethyl-phenoxy)-heptanoic acid;
(3S,5S)-3-Aminomethyl-5-methyl-7-(3-trifluoromethyl-phenoxy)-heptanoic acid;
(3S,5S)-3-Aminomethyl-5-methyl-7-(2-trifluoromethyl-phenoxy)-heptanoic acid;
(3S,5S)-3-Aminomethyl-5-methyl-7-(4-nitro-phenoxy)-heptanoic acid;
(3S,5S)-3-Aminomethyl-5-methyl-7-(3-nitro-phenoxy)-heptanoic acid;
(3S,5S)-3-Aminomethyl-5-methyl-7-(2-nitro-phenoxy)-heptanoic acid;
(3S,5S)-3-Aminomethyl-5-methyl-6-phenyl-hexanoic acid;
(3S,5S)-3-Aminomethyl-6-(4-chloro-phenyl)-5-methyl-hexanoic acid;
(3S,5S)-3-Aminomethyl-6-(3-chloro-phenyl)-5-methyl-hexanoic acid;
(3S,5S)-3-Aminomethyl-6-(2-chloro-phenyl)-5-methyl-hexanoic acid;
(3S,5S)-3-Aminomethyl-6-(4-methoxy-phenyl)-5-methyl-hexanoic acid;
(3S,5S)-3-Aminomethyl-6-(3-methoxy-phenyl)-5-methyl-hexanoic acid;
(3S,5S)-3-Aminomethyl-6-(2-methoxy-phenyl)-5-methyl-hexanoic acid;
(3S,5S)-3-Aminomethyl-6-(4-fluoro-phenyl)-5-methyl-hexanoic acid;
(3S,5S)-3-Aminomethyl-6-(3-fluoro-phenyl)-5-methyl-hexanoic acid;
(3S,5S)-3-Aminomethyl-6-(2-fluoro-phenyl)-5-methyl-hexanoic acid;
(3S,5R)-3-Aminomethyl-5-methyl-7-phenyl-heptanoic acid;
(3S,5R)-3-Aminomethyl-7-(4-chloro-phenyl)-5-methyl-heptanoic acid;
(3S,5R)-3-Aminomethyl-7-(3-chloro-phenyl)-5-methyl-heptanoic acid;
(3S,5R)-3-Aminomethyl-7-(2-chloro-phenyl)-5-methyl-heptanoic acid;
(3S,5R)-3-Aminomethyl-7-(4-methoxy-phenyl)-5-methyl-heptanoic acid;
(3S,5R)-3-Aminomethyl-7-(3-methoxy-phenyl)-5-methyl-heptanoic acid;
(3S,5R)-3-Aminomethyl-7-(2-methoxy-phenyl)-5-methyl-heptanoic acid;
(3S,5R)-3-Aminomethyl-7-(4-fluoro-phenyl)-5-methyl-heptanoic acid;
(3S,5R)-3-Aminomethyl-7-(3-fluoro-phenyl)-5-methyl-heptanoic acid;
(3S,5R)-3-Aminomethyl-7-(2-fluoro-phenyl)-5-methyl-heptanoic acid;
(3S,5R)-3-Aminomethyl-5-methyl-oct-7-enoic acid;
(3S,5R)-3-Aminomethyl-5-methyl-non-8-enoic acid;
(E)-(3S,5S)-3-Aminomethyl-5-methyl-oct-6-enoic acid;
(Z)-(3S,5S)-3-Aminomethyl-5-methyl-oct-6-enoic acid;
(Z)-(3S,5S)-3-Aminomethyl-5-methyl-non-6-enoic acid;
(E)-(3S,5S)-3-Aminomethyl-5-methyl-non-6-enoic acid;
(E)-(3S,5R)-3-Aminomethyl-5-methyl-non-7-enoic acid;
(Z)-(3S,5R)-3-Aminomethyl-5-methyl-non-7-enoic acid;
(Z)-(3S,5R)-3-Aminomethyl-5-methyl-dec-7-enoic acid;
(E)-(3S,5R)-3-Aminomethyl-5-methyl-undec-7-enoic acid;
(3S,5S)-3-Aminomethyl-5,6,6-trimethyl-heptanoic acid;
(3S,5S)-3-Aminomethyl-5,6-dimethyl-heptanoic acid;
(3S,5S)-3-Aminomethyl-5-cyclopropyl-hexanoic acid;
(3S,5S)-3-Aminomethyl-5-cyclobutyl-hexanoic acid;
(3S,5S)-3-Aminomethyl-5-cyclopentyl-hexanoic acid; and
(3S,5S)-3-Aminomethyl-5-cyclohexyl-hexanoic acid.

Still another more preferred embodiment of the invention method utilizes a compound of Formula IV selected from:
(3S,5R)-3-Aminomethyl-5-methyl-heptanoic acid;
(3S,5R)-3-Aminomethyl-5-methyl-octanoic acid;
(3S,5R)-3-Aminomethyl-5-methyl-nonanoic acid;
(3S,5R)-3-Aminomethyl-5-methyl-decanoic acid;
(3S,5R)-3-Aminomethyl-5-methyl-undecanoic acid;
(3S,5R)-3-Aminomethyl-5-methyl-dodecanoic acid;
(3S,5R)-3-Aminomethyl-5,9-dimethyl-decanoic acid;
(3S,5R)-3-Aminomethyl-5,7-dimethyl-octanoic acid;
(3S,5R)-3-Aminomethyl-5,8-dimethyl-nonanoic acid;
(3S,5R)-3-Aminomethyl-6-cyclopropyl-5-methyl-hexanoic acid;
(3S,5R)-3-Aminomethyl-6-cyclobutyl-5-methyl-hexanoic acid;
(3S,5R)-3-Aminomethyl-6-cyclopentyl-5-methyl-hexanoic acid;
(3S,5R)-3-Aminomethyl-6-cyclohexyl-5-methyl-hexanoic acid;
(3S,5R)-3-Aminomethyl-7-cyclopropyl-5-methyl-heptanoic acid;
(3S,5R)-3-Aminomethyl-7-cyclobutyl-5-methyl-heptanoic acid;
(3S,5R)-3-Aminomethyl-7-cyclopentyl-5-methyl-heptanoic acid;
(3S,5R)-3-Aminomethyl-7-cyclohexyl-5-methyl-heptanoic acid;
(3S,5R)-3-Aminomethyl-8-cyclopropyl-5-methyl-octanoic acid;
(3S,5R)-3-Aminomethyl-8-cyclobutyl-5-methyl-octanoic acid;
(3S,5R)-3-Aminomethyl-8-cyclopentyl-5-methyl-octanoic acid;
(3S,5R)-3-Aminomethyl-8-cyclohexyl-5-methyl-octanoic acid;
(3S,5S)-3-Aminomethyl-6-fluoro-5-methyl-hexanoic acid;
(3S,5S)-3-Aminomethyl-7-fluoro-5-methyl-heptanoic acid;
(3S,5R)-3-Aminomethyl-8-fluoro-5-methyl-octanoic acid;
(3S,5R)-3-Aminomethyl-9-fluoro-5-methyl-nonanoic acid;
(3S,5S)-3-Aminomethyl-7,7,7-trifluoro-5-methyl-heptanoic acid;
(3S,5R)-3-Aminomethyl-8,8,8-trifluoro-5-methyl-octanoic acid;
(3S,5R)-3-Aminomethyl-5-methyl-8-phenyl-octanoic acid;
(3S,5S)-3-Aminomethyl-5-methyl-6-phenyl-hexanoic acid; and
(3S,5R)-3-Aminomethyl-5-methyl-7-phenyl-heptanoic acid.

Another embodiment utilizes an A2D ligand that is a compound of the Formula V, VI, VII, or VIII 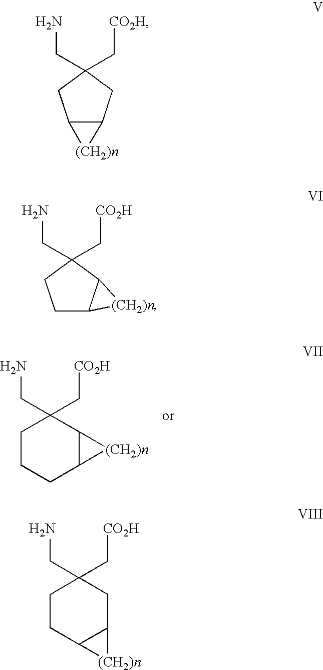
or a pharmaceutically acceptable salt thereof, wherein n is integer of from 1 to 4, where there are stereocenters, each center may be independently R or S.

A preferred embodiment of the invention method utilizes a compound of the Formula V, VI, VII, or VIII, wherein n is an integer of from 2 to 4.

Another preferred embodiment of the invention method utilizes a compound of the Formula V.

A still more preferred embodiment of the invention method utilizes a compound of the Formula V, VI, VII, or VIII that is selected from:
(1α,6α,8α)(2-Aminomethyl-octahydro-inden-2-yl)-acetic acid;
(2-Aminomethyl-octahydro-inden-2-yl)-acetic acid;
(2-Aminomethyl-octahydro-pentalen-2-yl)-acetic acid;
(2-Aminomethyl-octahydro-pentalen-2-yl)-acetic acid;
(3-Aminomethyl-bicyclo[3.2.0]hept-3-yl)-acetic acid;
(3-Aminomethyl-bicyclo[3.2.0]hept-3-yl)-acetic acid; and
(2-Aminomethyl-octahydro-inden-2-yl)-acetic acid;

Another still more preferred embodiment of the invention method utilizes a compound of the Formula V, VI, VII, or VIII that is selected from:
(1α,5β)(3-Aminomethyl-bicyclo[3.1.0]hex-3-yl)-acetic acid,
(1α,5β)(3-Aminomethyl-bicyclo[3.2.0]hept-3-yl)-acetic acid,
(1α,5β)(2-Aminomethyl-octahydro-pentalen-2-yl)-acetic acid,
(1α,6β)(2-Aminomethyl-octahydro-inden-2-yl)-acetic acid,
(1α,7β)(2-Aminomethyl-decahydro-azulen-2-yl)-acetic acid,
(1α,5β)(3-Aminomethyl-bicyclo[3.1.0]hex-3-yl)-acetic acid,
(1α,5β)(3-Aminomethyl-bicyclo[3.2.0]hept-3-yl)-acetic acid,
(1α,5β)(2-Aminomethyl-octahydro-pentalen-2-yl)-acetic acid,
(1α,6β)(2-Aminomethyl-octahydro-inden-2-yl)-acetic acid,
(1α,7β)(2-Aminomethyl-decahydro-azulen-2-yl)-acetic acid,
(1α,3α,5α)(3-Aminomethyl-bicyclo[3.1.0]hex-3-yl)-acetic acid,
(1α,3α,5α)(2-Aminomethyl-octahydro-pentalen-2-yl)-acetic acid,
(1α,6α,8α)(2-Aminomethyl-octahydro-inden-2-yl)-acetic acid,
(1α,7α,9α)(2-Aminomethyl-decahydro-azulen-2-yl)-acetic acid,
(1α,3β,5α)(3-Aminomethyl-bicyclo[3.1.0]hex-3-yl)-acetic acid,
(1α,3β,5α)(3-Aminomethyl-bicyclo[3.2.0]hept-3-yl)-acetic acid,
(1α,3β,5α)(2-Aminomethyl-octahydro-pentalen-2-yl)-acetic acid,
(1α,6α,8β)(2-Aminomethyl-octahydro-inden-2-yl)-acetic acid,
(1α,7α,9β)(2-Aminomethyl-decahydro-azulen-2-yl)-acetic acid,
((1R,3R,6R)-3-Aminomethyl-bicyclo[4.1.0]hept-3-yl)-acetic acid,
((1R,3S,6R)-3-Aminomethyl-bicyclo[4.1.0]hept-3-yl)-acetic acid,
((1S,3S,6S)-3-Aminomethyl-bicyclo[4.1.0]hept-3-yl)-acetic acid,
((1S,3R,6S)-3-Aminomethyl-bicyclo[4.1.0]hept-3-yl)-acetic acid,
((1R,3R,6S)-3-Aminomethyl-bicyclo[4.2.0]oct-3-yl)-acetic acid,
((1R,3S,6S)-3-Aminomethyl-bicyclo[4.2.0]oct-3-yl)-acetic acid,
((1S,3S,6R)-3-Aminomethyl-bicyclo[4.2.0]oct-3-yl)-acetic acid,
((1S,3R,6R)-3-Aminomethyl-bicyclo[4.2.0]oct-3-yl)-acetic acid,
((3αR,5R,7αS)-5-Aminomethyl-octahydro-inden-5-yl)-acetic acid,
((3αR,5S,7αS)-5-Aminomethyl-octahydro-inden-5-yl)-acetic acid,
((3αS,5S,7αR)-5-Aminomethyl-octahydro-inden-5-yl)-acetic acid,
((3αS,5R,7αR)-5-Aminomethyl-octahydro-inden-5-yl)-acetic acid,
((2R,4αS,8αR)-2-Aminomethyl-decahydro-naphthalen-2-yl)-acetic acid,
((2S,4αS,8αR)-2-Aminomethyl-decahydro-naphthalen-2-yl)-acetic acid,
((2S,4αR,8αS)-2-Aminomethyl-decahydro-naphthalen-2-yl)-acetic acid,
((2R,4αR,8αS)-2-Aminomethyl-decahydro-naphthalen-2-yl)-acetic acid,
((2R,4αS,9αR)-2-Aminomethyl-decahydro-benzocyclophepten-2-yl)-acetic acid,
((2S,4αS,9αR)-2-Aminomethyl-decahydro-benzocyclophepten-2-yl)-acetic acid,
((2S,4αR,9αS)-2-Aminomethyl-decahydro-benzocyclophepten-2-yl)-acetic acid,
((2R,4αR,9αS)-2-Aminomethyl-decahydro-benzocyclophepten-2-yl)-acetic acid,
((1R,3R,6S)-3-Aminomethyl-bicyclo[4.1.0]hept-3-yl)-acetic acid,
((1R,3S,6S)-3-Aminomethyl-bicyclo[4.1.0]hept-3-yl)-acetic acid,
((1S,3S,6R)-3-Aminomethyl-bicyclo[4.1.0]hept-3-yl)-acetic acid,
((1S,3R,6R)-3-Aminomethyl-bicyclo[4.1.0]hept-3-yl)-acetic acid,
((1R,3R,6R)-3-Aminomethyl-bicyclo[4.2.0]oct-3-yl)-acetic acid,
((1R,3S,6R)-3-Aminomethyl-bicyclo[4.2.0]oct-3-yl)-acetic acid,
((1S,3S,6S)-3-Aminomethyl-bicyclo[4.2.0]oct-3-yl)-acetic acid,
((1S,3R,6S)-3-Aminomethyl-bicyclo[4.2.0]oct-3-yl)-acetic acid,
((3αR,5R,7αR)-5-Aminomethyl-octahydro-inden-5-yl)-acetic acid,
((3αR,5S,7αR)-5-Aminomethyl-octahydro-inden-5-yl)-acetic acid,
((3αS,5S,7αS)-5-Aminomethyl-octahydro-inden-5-yl)-acetic acid,
((3αS,5R,7αS)-5-Aminomethyl-octahydro-inden-5-yl)-acetic acid,
((2R,4αR,8αR)-2-Aminomethyl-decahydro-naphthalen-2-yl)-acetic acid,
((2S,4αS,8αR)-2-Aminomethyl-decahydro-naphthalen-2-yl)-acetic acid,
((2S,4αR,8αS)-2-Aminomethyl-decahydro-naphthalen-2-yl)-acetic acid,
((2R,4αS,8αS)-2-Aminomethyl-decahydro-naphthalen-2-yl)-acetic acid,
((2R,4αR,9αR)-2-Aminomethyl-decahydro-benzocyclophepten-2-yl)-acetic acid,
((2S,4αR,9αR)-2-Aminomethyl-decahydro-benzocyclophepten-2-yl)-acetic acid,
((2S,4αS,9αS)-2-Aminomethyl-decahydro-benzocyclophepten-2-yl)-acetic acid, and
((2R,4αS,9αS)-2-Aminomethyl-decahydro-benzocyclophepten-2-yl)-acetic acid.

A more preferred embodiment of the invention method utilizes an A2D ligand of the Formula V, VI, VII, or VIII that is (1α,3α,5α)(3-aminomethyl-bicyclo[3.2.0]hept-3-yl)-acetic acid, or a pharmaceutically acceptable salt thereof.

A still more preferred embodiment of the invention method utilizes an A2D ligand of the Formula V, VI, VII, or VIII that is (1α,3α,5α)(3-aminomethyl-bicyclo[3.2.0]hept-3-yl)-acetic acid hydrochloride.

Other preferred embodiments of the invention method are those wherein the A2D ligand that is employed is selected from the following compounds and their pharmaceutically acceptable salts:
3-(1-aminomethyl-cyclohexylmethyl)-4H-[1,2,4]oxadiazol-5-one;
(S,S)-(1-aminomethyl-3,4-dimethyl-cyclopentyl)-acetic acid;
(R,S)-3-aminomethyl-5-methyl-octanoic acid;
(S,R)-3-aminomethyl-5-methyl-octanoic acid;
(3-aminomethyl-bicyclo[3.2.0]hept-3-yl)-acetic acid;
(3-aminomethyl-bicyclo[3.2.0]hept-3-yl)-acetic acid, wherein the cyclobutyl ring is trans to the methylamine group; and
C-[1-(1H-tetrazol-5-ylmethyl)-cycloheptyl]-methylamine.

These compounds can be prepared as described below or in PCT Patent Application WO 99/21824, published May 6, 1999, PCT Patent Application WO 00/76958, published Dec. 21, 2000, or PCT Patent Application WO 01/28978, published Apr. 26, 2001. These applications are incorporated herein by reference in their entireties.

A more preferred embodiment of the invention method utilizes the hydrochloride salt of the compound 3-(1-aminomethyl-cyclohexylmethyl)4H-[1,2,4]oxadiazol-5-one.

Another preferred embodiment of the invention method utilizes the cyclic amino acids of the Formula I. These are described in U.S. Pat. No. 4,024,175 and U.S. Pat. No. 4,087,544, which are both incorporated herein by reference in their entireties.

Another preferred embodiment of the invention method utilizes an A2D ligand of the Formula II, and these compounds are described in U.S. Pat. No. 5,563,175, which is incorporated herein by reference in its entirety.

Another preferred embodiment of the invention method utilizes an A2D ligand of the Formula III, IIIC, IIIF, IIIG, or IIIH. These compounds are described in PCT Patent Application No. WO 99/31075, which is incorporated herein by reference in its entirety.

Another preferred embodiment of the invention method utilizes an A2D ligand of the Formula IV, which are described in PCT Patent Application No. WO 00/76958, which is incorporated herein by reference in its entirety.

Other preferred A2D ligands to be utilized in the invention method are compounds of the Formula (IXA) and (IXB), which are described in PCT Patent Application No. WO 99/31074, which is incorporated herein by reference in its entirety.

PCT Patent Application No. WO 01/28978, which is incorporated herein by reference in its entirety, describes other preferred A2D ligands that can be utilized in preferred embodiments of the invention. Such compounds are compounds of the Formulas V, VI, VII, and VIII.

Another preferred embodiment of the invention method utilizes an A2D ligand which is a compound of the Formula (IXA) or (IXB) 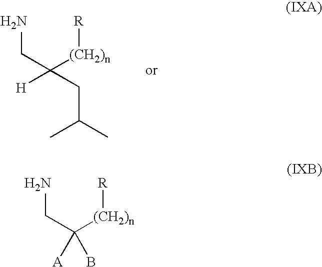
or a pharmaceutically acceptable salt thereof wherein:
n is an integer of from 0 to 2;
R is sulfonamide,
   amide,
   phosphonic acid,
   heterocycle,
   sulfonic acid, or
   hydroxamic acid;
A is hydrogen or methyl; and 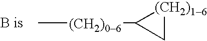
straight or branched alkyl of from 1 to 11 carbons, or
—(CH₂)₁₋₄₋Y—(CH₂)₀₋₄₋phenyl wherein Y is —O—, —S—, —NR′₃ wherein:
R′₃ is alkyl of from 1 to 6 carbons, cycloalkyl of from 3 to 8 carbons, benzyl or phenyl wherein benzyl or phenyl can be unsubstituted or substituted with from 1 to 3 substituents each independently selected from alkyl, alkoxy, halogen, hydroxy, carboxy, carboalkoxy, trifluoromethyl, and nitro.

A more preferred embodiment of the invention method utilizes an A2D ligand which is a compound of the Formula (IXA) or (IXB), wherein R is a sulfonamide selected from —NHSO₂R¹⁵ and —SO₂NHR¹⁵, wherein R¹⁵ is straight or branched alkyl or trifluoromethyl.

An especially preferred embodiment of the invention method utilizes a compound of the Formula (IXA) or (IXB) selected from:
4-Methyl-2-(1H-tetrazol-5-ylmethyl)-pentylamine;
3-(2-Aminomethyl-4-methyl-pentyl)-4H-[1,2,4]oxadiazole-5-thione, HCl;
(2-Aminomethyl-4-methyl-pentyl)-phosphonic acid;
3-(3-Amino-2-cyclopentyl-propyl)-4H-[1,2,4]oxadiazol-5-one;
3-(3-Amino-2-cyclopentyl-propyl)-4H-[1,2,4]thiadiazol-5-one;
2-Cyclopentyl-3-(2-oxo-2,3-dihydro-2λ⁴-[1,2,3 ,5]oxathiadiazol-4-yl)-propylamine;
3-(3-Amino-2-cyclobutyl-propyl)-4H-[1,2,4]oxadiazol-5-one;
3-(3-Amino-2-cyclobutyl-propyl)-4H-[1,2,4]thiadiazol-5-one; and
2-Cyclobutyl-3-(2-oxo-2,3-dihydro-2λ⁴-[1,2,3,5]oxathiadiazol-4-yl)-propylamine.

Another preferred embodiment of the invention method utilizes a compound of the Formula (IXA) or (IXB), wherein R is a phosphonic acid, —PO₃H₂.

Another preferred embodiment of the invention method utilizes a compound of the Formula (IXA) or (IXB), wherein R is

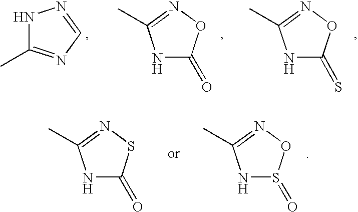

More preferred is an embodiment of the invention method that utilizes a compound of the Formula (IXA) or (IXB), wherein R is

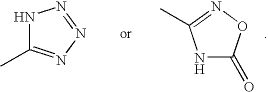

Still more preferred is an embodiment of the invention method that utilizes a compound of the Formula (IXA) or (IXB) that is 3-(2-aminomethyl-4-methyl-pentyl)-4H-[1,3,4]oxadiazol-5-one, or a pharmaceutically acceptable salt thereof.

Still more preferred is an embodiment of the invention method that utilizes a compound of the Formula (IXA) or (IXB) that is 3-(2-aminomethyl-4-methyl-pentyl)-4H-[1,2,4]oxadiazol-5-one hydrochloride.

Other A2D ligands that can be utilized in preferred embodiments of the invention method are described in U.S. Ser. No. 10/401,060, filed on Mar. 27, 2003.Such A2D ligands are compounds of the formulas X, XA, XB, XI, XIA, XIB and XB-1, as described below. Compounds of the formula X have the formula 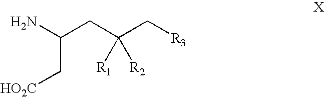
wherein R₁ is hydrogen or (C₁-C₃)alkyl optionally substituted with from one to five fluorine atoms;
R₂ is hydrogen or (C₁-C₃)alkyl optionally substituted with from one to five fluorine atoms;
R₃ is (C₁-C₆)alkyl, (C₃-C₆)cycloalkyl, (C₃-C₆)cycloalkyl-(C₁-C₃)alkyl, phenyl, phenyl-(C₁-C₃)alkyl, pyridyl, pyridyl-(C₁-C₃)alkyl, phenyl-N(H)—, or pyridyl-N(H)—, wherein each of the foregoing alkyl moieties can be optionally substituted with from one to five fluorine atoms, preferably with from zero to three fluorine atoms, and wherein said phenyl and said pyridyl and the phenyl and pyridyl moieties of said phenyl-(C₁-C₃)alkyl and said pyridyl-(C₁-C₃)alkyl, respectively, can be optionally substituted with from one to three substituents, preferably with from zero to two substituents, independently selected from chloro, fluoro, amino, nitro, cyano, (C₁-C₃)alkylamino, (C₁-C₃)alkyl optionally substituted with from one to three fluorine atoms and (C₁-C₃)alkoxy optionally substituted with from one to three fluorine atoms;
with the proviso that when R₁ is hydrogen, R₂ is not hydrogen; and the pharmaceutically acceptable salts of such compounds.

Compounds of the formula XI have the formula 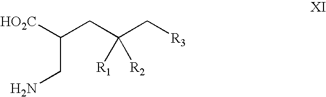
wherein R₁, R₂, and R₃ are defined as above, and the pharmaceutically acceptable salts of such compounds.

Compounds of the formula XA have the formula 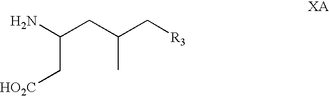
wherein R₃ is defined as above, and the pharmaceutically acceptable salts of such compounds.

Compounds of the formula XIA have the formula 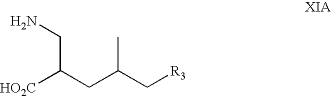
wherein R₃ is defined as above, and the pharmaceutically acceptable salts of such compounds.

Compounds of the formula XIB have the formula 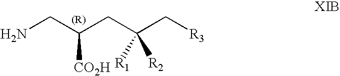
wherein R₁, R₂, and R₃ are defined as above.

Compounds of the formula XB have the formula 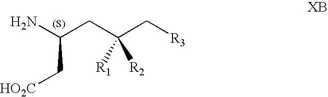
wherein R₁, R₂, and R₃ are defined as above.

Compounds of the formula XB-1 have the formula 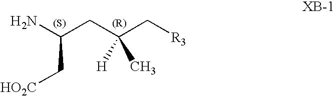
wherein R₃ is defined as above.

Other A2D ligands that can be used in preferred embodiments of the present invention method are described in PCT Patent Application No. WO 99/31057, which is incorporated herein by reference in its entirety. Such A2D ligands are compounds of the Formulas (XII) and (XEi) 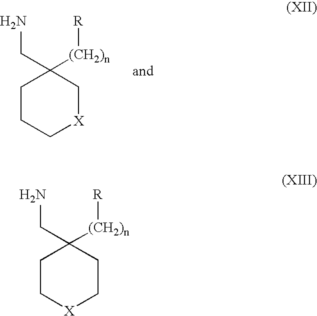
or a pharmaceutically acceptable salt thereof wherein:
n is an integer of from 0 to 2;
R is sulfonamide,
   amide,
   phosphonic acid,
   heterocycle,
   sulfonic acid, or
   hydroxamic acid; and
X is —O—, —S—, —S(O)—, —S(O)₂—, or NR′₁ wherein R′₁ is hydrogen, straight or branched alkyl of from 1 to 6 carbons, benzyl, —C(O)R′₂ wherein R′₂ is straight or branched alkyl of 1 to 6 carbons, benzyl or phenyl or —CO₂R′₃ wherein R′₃ is straight or branched alkyl of from 1 to 6 carbons, or benzyl wherein the benzyl or phenyl groups can be unsubstituted or substituted by from 1 to 3 substituents selected from halogen, trifluoromethyl, and nitro.

Other A2D ligands that may be utilized in preferred embodiments of the invention method are described in PCT Patent Application No. WO 98/17627, which is incorporated herein by reference in its entirety. Such A2D ligands are compounds of the formula 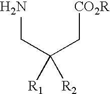
or a pharmaceutically acceptable salt thereof wherein:
R is hydrogen or lower alkyl;
R₁ is hydrogen or lower alkyl; 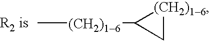
straight or branched alkyl of from 7 to 11 carbon atoms, or
—(CH₂)₍₁₋₄₎—X—(CH₂)₍₀₋₄₎-phenyl wherein
X is —O—, —S—, —NR₃ wherein
R₃ is alkyl of from 1 to 6 carbons, cycloalkyl of from 3 to 8 carbons, benzyl or phenyl;
wherein phenyl and benzyl can be unsubstituted or substituted with from 1 to 3 substituents each independently selected from alkyl, alkoxy, halogen, hydroxy, carboxy, carboalkoxy, trifluoromethyl, amino, and nitro.

Other A2D ligands that can be utilized in preferred embodiments of the invention method are described in PCT Patent Application No. WO 99/61424, which is incorporated herein by reference in its entirety. Such A2D ligands are compounds of the formulas (1), (2), (3), (4), (5), (6), (7), and (8) 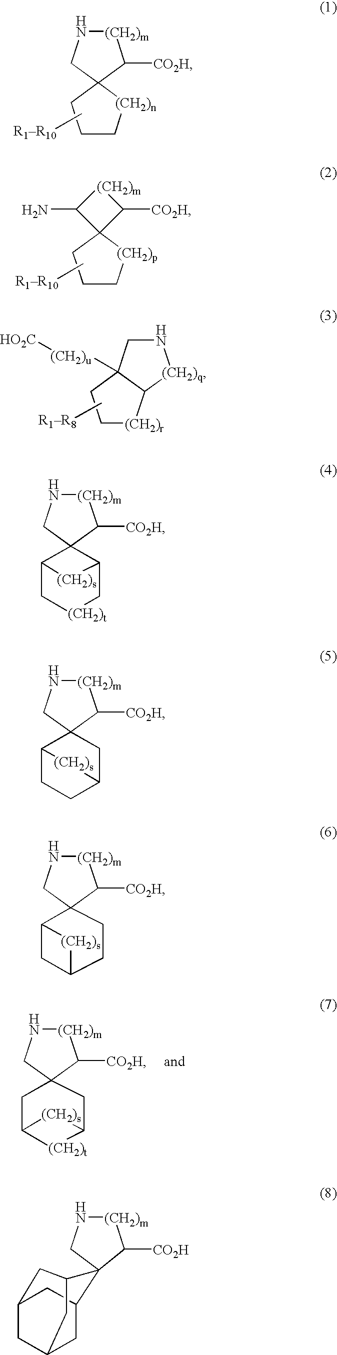
and the pharmaceutically acceptable salts and prodrugs of such compounds wherein:
R₁ to R₁₀ are each independently selected from hydrogen or a straight or branched alkyl of from 1 to 6 carbons, benzyl, or phenyl;
m is an integer of from 0 to 3;
n is an integer of from 1 to 2;
o is an integer of from 0 to 3;
p is an integer of from 1 to 2;
q is an integer of from 0 to 2;
r is an integer of from 1 to 2;
s is an integer of from 1 to 3;
t is an integer of from 0 to 2; and
u is an integer of from 0 to 1.

All U.S. patents and WO publications referenced above are incorporated herein by reference in their entireties.

It should be appreciated that the terms “uses”, “utilizes”, and “employs” are used interchangeably when describing an embodiment of the present invention.

The phrase “lower alkyl” means a straight or branched alkyl group or radical having from 1 to 6 carbon atoms, and includes methyl, ethyl, n-propyl, i-propyl, n-butyl, i-butyl, sec-butyl, tert-butyl, n-pentyl, n-hexyl, and the like.

The term “alkyl” is a straight or branched group of from 1 to 8 carbon atoms, unless stated otherwise, including but not limited to methyl, ethyl, propyl, n-propyl, isopropyl, butyl, 2-butyl, tert-butyl, and octyl. Alkyl can be unsubstituted or substituted by hydroxy or from 1 to 3 fluorine atoms. Preferred groups are methyl and ethyl.

The term “alkenyl” is a straight or branched group of from 2 to 8 carbon atoms containing 1 or 2 or 3 double bonds including but not limited to ethenyl, propen-1-yl, propen-2-yl, propen-3-yl, 1-hexen-3-yl, and hept-1,3-dien-7-yl. Alkenyl can be unsubstituted or substituted by from 1 to 3 fluorine atoms.

The term “cycloalkyl” means a cyclic group of from 3 to 7 carbon atoms including but not limited to cyclopropyl, cyclobutyl, and cycloheptyl.

The benzyl and phenyl groups may be unsubstituted or substituted with from 1 to 3 groups each independently selected from halogen, especially fluoro, alkoxy, alkyl, and NH₂.

“Halogen” includes fluorine, chlorine, bromine, and iodine.

The term “alkoxy” means the group —O-alkyl wherein alkyl is as defined above.

Sulfonamides are those of formula —NHSO₂R¹⁵ or —SO2NHR15 wherein R¹⁵ is a straight or branched alkyl group of from 1 to 6 carbons or a trifluoromethyl.

Amides are compounds of formula —NHCOR¹² wherein R¹² is straight or branched alkyl of from 1 to 6 carbons, benzyl, and phenyl.

Phosphonic acids are —PO₃H₂.

Sulfonic acids are —SO₃H.

Hydroxamic acid is

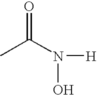

Heterocycles are groups of from 1 to 2 rings, the monocyclic rings having from 4 to 7 ring members and the bicyclic ring having from 7 to 12 ring members, with from 1 to 6 heteroatoms selected from oxygen, nitrogen, and sulfur.

Preferred heterocycles are

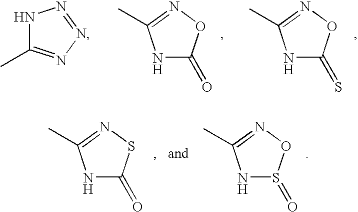

The term “alkyl” is a straight or branched group of from 1 to 11 carbon atoms including but not limited to methyl, ethyl, propyl, n-propyl, isopropyl, butyl, 2-butyl, tert-butyl, pentyl, hexyl, and n-hexyl, heptyl, octyl, nonyl, decyl, and undecyl except as where otherwise stated.

The cycloalkyl groups are from 3 to 8 carbons and are cyclopropyl, cyclobutyl, cyclopentyl, cyclohexyl, cycloheptyl, and cyclooctyl unless otherwise stated.

The benzyl and phenyl groups may be unsubstituted or substituted by from 1 to 3 substituents selected from hydroxy, carboxy, carboalkoxy, halogen, CF₃, nitro, alkyl, and alkoxy. Preferred are fluorine and chlorine.

Carboalkoxy is —COOalkyl wherein alkyl is as described above. Preferred are carbomethoxy and carboethoxy.

Examples of preferred A2D ligands for use with the present invention are those compounds generally or specifically disclosed in U.S. 4,024,175, particularly gabapentin, EP641330, particularly pregabalin, U.S. 5,563,175, WO9733858, WO9733859, WO9931057, WO9931074, WO9729101, WO02085839, particularly [(1R,5R,6S)-6-(Aminomethyl)bicyclo[3.2.0]hept-6-yl]acetic acid, WO9931075, particularly 3-(1-Aminomethyl-cyclohexylmethyl)-4H-[1,2,4]oxadiazol-5-one and C-[1-(1H-Tetrazol-5-ylmethyl)-cycloheptyl]-methylamine, WO9921824, particularly (3S,4S)-(1-Aminomethyl-3,4-dimethyl-cyclopentyl)-acetic acid, WO0190052, WO0128978, particularly (1α,3α,5α)(3-amino-methyl-bicyclo[3.2.0]hept-3-yl)-acetic acid, EP0641330, WO9817627, WO0076958, WPO3/082807A2, particularly, (3S,5R)-3-aminomethyl-5-methyl-octanoic acid, (3S,5R)-3-amino-5-methyl-heptanoic acid, and (3S,5R)-3-amino-5-methyl-nonanoic acid, EP1 178034, EP1201240, WO9931074, WO03000642, WO0222568, WO0230871, WO0230881 WO02100392, WO02100347, WO0242414, WO0232736 and WO0228881, and pharmaceutically acceptable salts and solvates thereof.

Most preferred A2D ligands of the present invention include: gabapentin, pregabalin, [(1R,5R,6S)-6-(Aminomethyl)bicyclo[3.2.0]hept-6-yl]acetic acid, 3-(1-Aminomethyl-cyclohexylmethyl)-4H-[1,2,4]oxadiazol-5-one and C-[1-(1H-Tetrazol-5-ylmethyl)-cycloheptyl]-methylamine, (3S,4S)-(1-Aminomethyl-3,4-dimethyl-cyclopentyl)-acetic acid, (1α,3α,5α)(3-amino-methyl-bicyclo[3.2.0]hept-3-yl)-acetic acid, (3S,5R)-3-Aminomethyl-5-methyl-octanoic acid, (3S,5R)-3-amino-5-methyl-heptanoic acid, and (3S,5R)-3-amino-5-methyl-nonanoic acid.

SSRI's useful for the methods and pharmaceutical compositions of the present invention include, but are not limited to sertraline (Zoloft®), sertraline metabolite demethylsertraline, fluoxetine (Prozac®), norfluoxetine (fluoxetine desmethyl metabolite), fluvoxamine (Luvox®), paroxetine (Seroxat®, Paxil®) and its alternative formulation, Paxil-CR®, citalopram (Celexa®), citalopram metabolite desmethylcitalopram, escitalopram (Lexapro®), d,l-fenfluramine (Pondimin®), femoxetine, ifoxetine, cyanodothiepin, litoxetine, cericlamine, dapoxetine, nefazaodone (Serxone®), and trazodone (Desyrel®), or any prodrug thereof or any pharmaceutically acceptable salt of said SSRI or said prodrug. Preferably, the SSRI is sertraline.

SNRI's useful for the methods and pharmaceutical compositions of the present invention include, but are not limited to, reboxetine (Edronax®) and all enantiomers of reboxetine, ie., (R/R,S/S,R/S,SIR), desipramine (Norpramin®), maprotiline (Ludiomil®), lofepramine (Gamanil®), mirtazepine (Remeron®), oxaprotiline, fezolamine, atomoxetine and buproprion (Wellbutrin®), buproprion metabolite hydroxybuproprion, nomifensine (Merital®), viloxazine (Vivalan®), or mianserin (Bolvidon®) or any prodrug thereof or any pharmaceutically acceptable salt of said SNRI or said prodrug. Preferably, the SNRI is reboxetine.

Pharmaceutical agents which inhibit the reuptake of both serotonin and norepinephrine include venlafaxine (Effexor®), venlafaxine metabolite O-desmethylvenlafaxine, clomipramine (Anafranil®), clomipramine metabolite desmethylclomipramine, duloxetine (Cymbalta®), milnacipran, and imipramine (Tofranil® or Janimine®).

Other SSRI's useful for the methods and pharmaceutical compositions of the present invention include the cis-isomeric compound of the formula 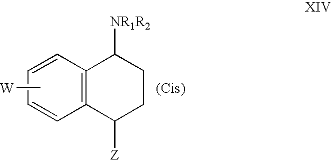
wherein with regard to formula XIV R₁ is selected from the group consisting of hydrogen and normal alkyl of from 1 to 3 carbon atoms, R₂ is normal alkyl of from 1 to 3 carbon atoms, Z is 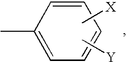
X and Y are each selected from the group consisting of hydrogen, fluoro, chloro, bromo, trifluoromethyl, alkoxy of from 1 to 3 carbon atoms and cyano, with at least one of X and Y being other than hydrogen, and W is selected from the group consisting of hydrogen, fluoro, chloro, bromo, trifluoromethyl and alkoxy of from 1 to 3 carbon atoms and wherein the term “cis-isomeric” refers to the relative orientation of the NRIR₂ and Z moieties on the cyclohexene ring with said compound being either the (1S)-enantiomer or the racemic mixture of the (1S)-enantiomer with the corresponding (1R)-enantiomer or a prodrug thereof or a pharmaceutically acceptable salt thereof or of said prodrug.

Other SNRI's useful for the methods and pharmaceutical compositions of the present invention include the racemates and optical isomers corresponding to a compound of the formula 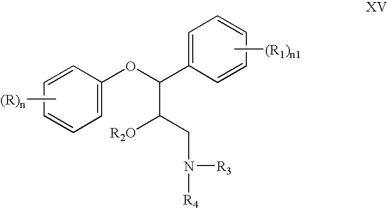
preferably the substituted propanolamine and morpholine derivatives, corresponding to the above SNRI formula XV, wherein
n and n₁ are, independently, 1, 2 or 3;
each of the groups R and R₁, which may be the same or different, is hydrogen; halogen; halo-C₁-C₆ alkyl; hydroxy; C₁-C₆ alkoxy; C₁-C₆ alkyl optionally substituted; aryl-C₁-C₆ alkyl optionally substituted; aryl-C₁-C₆ alkoxy optionally substituted; —NO₂; 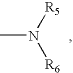
   wherein
R₅ and R6 are, independently, hydrogen or C₁-C₆ alkyl, or two adjacent R groups or two adjacent R₁ groups, taken together, form the —O—CH₂—O— radical; R₂ is hydrogen; C₁-C₁₂ alkyl optionally substituted, or aryl-C₁-C₆ alkyl; each of the groups R₃ and R4, which may be identical or different, is hydrogen, C₁-C₆ alkyl optionally substituted, C₂-C₄ alkenyl, C₂-C₄ alkynyl, aryl-C₂-C₄ alkyl optionally substituted, C₃-C₇ cycloalkyl optionally substituted, or R₃ and R₄ with the nitrogen atom to which they are bonded form a pentatomic or hexatomic saturated or unsaturated, optionally substituted, heteromonocyclic radical optionally containing other heteroatoms belonging to the class of O, S and N; or R₂ and R₄, taken together, form the —CH₂—CH₂— radical. This invention also includes the pharmaceutically acceptable salts of compounds with formula (XV) as well as all the possible isomers and their mixtures, the metabolites provided with pharmacological, e.g. antidepressant, activity and the metabolic precursors of the compounds with formula (XV). The alkyl, alkenyl, alkynyl and alkoxy groups may be straight or branched chains.

When one or more of the groups R and R₁ is a substituted C₁-C₆ alkyl group it is preferably C₁-C₆ alkyl substituted by one or more substituents chosen from hydroxy, C₁-C₆ alkoxy, 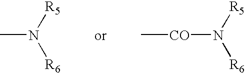
in which R₅ and R₆ are as defined above. An aryl group is preferably phenyl.

When one or more of the groups R₃ and R₄ is a substituted C₁-C₆ alkyl group, it is preferably C₁-C₆ alkyl substituted by one or more substituents chosen from halogen, hydroxy, C₁-C₆ alkoxy, 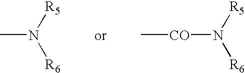
with R₅ and R₆ as defined above. The same substituents may be present on a substituted C₁-C₁₂ alkyl group.

Substituted aryl-C₁-C₆ alkyl, aryl-C₁-C₄ alkyl and aryl-C₁-C₆ alkoxy groups are preferably aryl-C₁-C₆ alkyl, aryl-C₁-C₄ alkyl and aryl-C₁-C₆ alkoxy groups in which the aryl group is substituted by one or more C₁-C₆ alkyl, halogen, halo-C₁-C₆ alkyl, hydroxy, C₁-C₆ alkoxy and 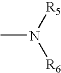
with R₅ and R₆ as defined above.

A substituted C₃-C₇ cycloalkyl group is a C₃-C₇ cycloalkyl substituted by one or more substituents preferably chosen from C₁-C₆ alkyl, halogen, halo-C₁-C₆-alkyl, hydroxy, C₁-C₆alkoxy and 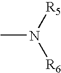
in which R₅ and R₆ are defined above.

A C₁-C₆ alkyl group is preferably methyl, ethyl or isopropyl.

A C₁-C₁₂ alkyl group is preferably methyl, ethyl, isopropyl or octyl.

A C₂-C₄ alkenyl group is preferably vinyl or allyl.

A C₂-C₄ alkynyl group is preferably propargyl.

A halo-C₁-C₆ alkyl group is preferably trihalo-C₁-C₆ alkyl, in particular trifluoromethyl.

A C₁-C₆ alkoxy group is preferably methoxy or ethoxy.

An aryl-C₁-C₆ alkyl or aryl-C₁-C₄ alkyl group is preferably benzyl or phenethyl.

An aryl-C₁-C₆ alkoxy group is preferably benzyloxy.

In a 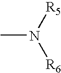
group, R₅ and R₆ preferably are, independently, hydrogen or C₁-C₃ alkyl, in particular methyl, ethyl or isopropyl.

A C₃-C₇ cycloalkyl group is preferably cyclopropyl, cyclopentyl or cyclohexyl.

When R₃ and R₄, with the nitrogen atom to which they are linked, form a substituted heteromonocyclic radical, the substituents are preferably C₁-C₆ alkyl or aryl, in particular methyl or phenyl; preferred heteromonocyclic radicals are morpholino, piperidino, N-pyrrolidinyl, N-methyl-piperazinyl and N-phenyl-piperazinyl.

When two adjacent R groups or two adjacent R₁ groups form the —O—CH₂—O— radical, this is preferably a 3,4-methylendioxy radical.

Owing to the presence of at least two asymmetric carbon atoms, for each compound of formula (XV) at least two distinct diastereoisomers may exist, from which at least four distinct enantiomers may be obtained: both the single diastereoisomers and their mixture as well as the single enantiomers are included in the object of this invention. Examples of pharmaceutically acceptable salts of compounds (XV) are both the salts with inorganic acids, for example hydrochloric acid, hydrobromic acid, sulphuric acid, and the salts with organic acids, for example, citric acid, tartaric acid, methansulphonic acid, fumaric acid, malic acid, maleic acid and mandelic acid.

According to this invention preferred salts of compounds (XV) are those in which the 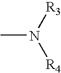
group is salified with one of the acids mentioned above, preferably the hydrochloric acid.

Further compounds useful for the methods and pharmaceutical compositions of the present invention include compounds that are both selective serotonin re-uptake inhibitors and norepinephrine/dopamine re-uptake inhibitors (SRI/DRI) and are of the formula 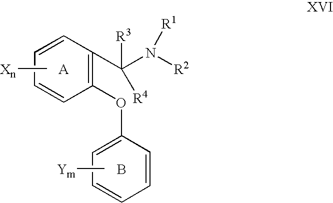
wherein phenyl ring A and phenyl ring B can each, independently, be replaced by a naphthyl group, and wherein when phenyl ring A is replaced by a naphthyl group, the ethereal oxygen of structure I and the carbon to which R³, R⁴ and NR¹R² are attached, are attached to adjacent ring carbon atoms of the naphthyl group and neither of said adjacent ring carbon atoms is also adjacent to a fused ring carbon atom of said naphthyl group;
n and m are, selected, independently, from one, two and three;
R¹ and R² are selected, independently, from hydrogen, (C₁-C₄)alkyl, (C₂-C₄)alkenyl, and (C₂-C₄)alkynyl, or R¹ and R², together with the nitrogen to which they are attached, form a four to eight membered saturated ring containing one or two heteroatoms, including the nitrogen to which R¹ and R² are attached, wherein the second heteroatom, when present, is selected from oxygen, nitrogen and sulfur, with the proviso that said ring can not contain two adjacent oxygen atoms or two adjacent sulfur atoms, and wherein said ring may optionally be substituted at available binding sites with from one to three substituents selected, independently, from hydroxy and (C₁-C₆)alkyl;
R³ and R⁴ are selected, independently, from hydrogen and (C₁-C₄) alkyl optionally substituted with from one to three fluorine atoms, or R³ and R⁴, together with the carbon to which they are attached, form a four to eight membered saturated carbocyclic ring, and wherein said ring may optionally be substituted at available binding sites with from one to three substituents selected, independently, from hydroxy and (C₁-C₆)alkyl;
or R² and R³, together with the nitrogen to which R² is attached and the carbon to which R³ is attached, form a four to eight membered saturated ring containing one or two heteroatoms, including the nitrogen to which R2 is attached, wherein the second heteroatom, when present, is selected from oxygen, nitrogen and sulfur, with the proviso that said ring can not contain two adjacent oxygen atoms or two adjacent sulfur atoms, and wherein said ring may optionally be substituted at available binding sites with from one to three substituents selected, independently, from hydroxy and (C₁-C₆)alkyl;
each X is selected, independently, from hydrogen, halo (ie., chloro, fluoro, bromo or iodo), (C₁-C₄)alkyl optionally substituted with from one to three fluorine atoms, (C₁-C₄)alkoxy optionally substituted with from one to three fluorine atoms, cyano, nitro, amino, (C₁-C₄)alkylamino, di-[(C₁-C₄)alkyl]amino, NR⁵(C═O)(C₁-C₄)alkyl, SO₂NR⁵R⁶ and SOₚ(C₁-C₆)alkyl, wherein R⁵ and R⁶ are selected, independently, from hydrogen and (C₁-C₆)alkyl, and p is zero, one or two; and
each Y is selected, independently, from hydrogen, (C₁-C₆)alkyl and halo;
with the proviso that: (a) no more than one of NR¹R², CR³R⁴ and R²NCR³ can form a ring; and (b) at least one X must be other than hydrogen when (i) R³ and R⁴ are both hydrogen, (ii) R¹ and R² are selected, independently, from hydrogen and (C₁-C₄)alkyl, and (iii) ring B is mono- or disubstituted with, respectively, one or two halo groups; and the pharmaceutically acceptable salts thereof. Compounds according to formula XVI are described in WO 00/50380.

In a further aspect, the present invention is directed to a method of treating a subject, including a mammal, and particularly a human, suffering from depression, or depression with concomitant anxiety, or depression with concomitant sleep disorders including insomnia, or depression with concomitant anxiety and sleep disorders including insomnia, or post traumatic stress disorder, comprising administering to the subject a therapeutically effective amount of:
(a) an A2D ligand corresponding to (i) a compound of formula I, wherein with regard to formula I, R₁, R₂ and R₃ are as defined above and including the racemate or the individual enantiomeric isomers thereof, or a prodrug thereof, or a pharmaceutically acceptable salt thereof or said prodrug; or (ii) a compound of formula II wherein with regard to formula II, R₁ and n are as defined above, or a prodrug thereof or a pharmaceutically acceptable salt thereof or of said prodrug; or a mixture thereof; and,
(b) an SSRI corresponding to a compound of formula III, wherein with regard to formula III R₁, R₂, X, Y and Z are as defined above and with said compound being either the (1S)-enantiomer or the racemic mixture of the (1S)-enantiomer with the corresponding (1R)-enantiomer or a prodrug thereof or a pharmaceutically acceptable salt thereof or of said prodrug, said compounds (a) and (b) being administered in either a sequential or concurrent manner, or
(c) an SNRI corresponding to a compound of formula IV, wherein with regard to formula IV R, R₁, R₂, R₃, R₄, R₅ and R₆ are as defined above and including the racemate or the individual enantiomeric isomers and diastereoisomers thereof, or a prodrug thereof, or a pharmaceutically acceptable salt thereof or of said prodrug, said compounds (a) and (c) being administered in either a sequential or concurrent manner, or
(d) a combination of (a) with (b) and (c).

In a still further aspect, the present invention is directed to a pharmaceutical composition that has efficacy in treating a subject, including a mammal, and particularly a human, suffering from depression and particularly depression combined with one or more of the aforesaid conditions, diseases or disorders, said pharmaceutical composition comprising a therapeutically effective amount of:
(a) an A2D ligand corresponding to: (i) a compound of formula I, wherein with regard to formula I, R₁, R₂ and R₃ are as defined above and including the racemate or the individual enantiomeric isomers thereof, or a prodrug thereof, or a pharmaceutically acceptable salt thereof or said prodrug; or (ii) a compound of formula II wherein with regard to formula II, R₁ and n are as defined above, or a prodrug thereof or a pharmaceutically acceptable salt thereof or of said prodrug; or a mixture thereof; and,
(b) an SSRI corresponding to a compound of formula XIV, wherein with regard to formula XIV R₁, R₂, X, Y and Z are as defined above and with said compound being either the (1S)-enantiomer or the racemic mixture of the (1S)-enantiomer with the corresponding (1R)-enantiomer or a prodrug thereof or a pharmaceutically acceptable salt thereof or of said prodrug; or,
(c) an SNRI corresponding to a compound of formula XV, wherein with regard to formula XV, R, R₁, R₂,R₃, R₄, R₅ and R₆ are as defined above and including the racemate or the individual enantiomeric isomers and diastereoisomers thereof, or a prodrug thereof, or a pharmaceutically acceptable salt thereof or of said prodrug; or
(d) a combination of (a) with (b) and (c) and, optionally, a pharmaceutically acceptable vehicle, carrier or diluent.

The term “treating”, as used herein, refers to reversing, alleviating, inhibiting the progress of, or preventing the disorder or condition to which such term applies, or preventing one or more symptoms of such condition or disorder. The term “treatment”, as used herein, refers to the act of treating, as “treating” is defined immediately above.

The compounds of the present combination invention can exist in unsolvated forms as well as solvated forms, including hydrated forms. In general, the solvated forms, including hydrated forms, which may contain isotopic substitutions (e.g. D2O, d6-acetone, d6-DMSO), are equivalent to unsolvated forms and are encompassed within the scope of the present invention.

Certain of the compounds of the present invention possess one or more chiral centers and each center may exist in the R or S configuration. The present invention includes all enantiomeric and epimeric forms as well as the appropriate mixtures thereof. Separation of diastereoisomers or cis and trans isomers may be achieved by conventional techniques, e.g. by fractional crystallisation, chromatography or H.P.L.C. of a stereoisomeric mixture of a compound of the invention or a suitable salt or derivative thereof.

A number of A2D ligands of the present invention are amino acids. Since amino acids are amphoteric, pharmacologically compatible salts can be salts of appropriate non-toxic inorganic or organic acids or bases. Suitable acid addition salts are the acetate, aspartate, benzoate, besylate, bicarbonate/carbonate, bisulphate, camsylate, citrate, edisylate, esylate, fumarate, gluceptate, gluconate, glucuronate, hibenzate, hydrochloride/chloride, hydrobromide/bromide, hydroiodide/iodide, hydrogen phosphate, isethionate, D- and L-lactate, malate, maleate, malonate, mesylate, methylsulphate, 2-napsylate, nicotinate, nitrate, orotate, palmoate, phosphate, saccharate, stearate, succinate sulphate, D- and L-tartrate, and tosylate salts. Suitable base salts are formed from bases which form non-toxic salts and examples are the sodium, potassium, aluminium, calcium, magnesium, zinc, choline, diolamine, olamine, arginine, glycine, tromethamine, benzathine, lysine, meglumine and diethylamine salts. Salts with quaternary ammonium ions can also be prepared with, for example, the tetramethyl-ammonium ion. The compounds of the invention may also be formed as a zwitterion. Furthermore, since a number of the SSRIs, SNRIs, and dual acceptable inhibitors of the present invention are amines and a number of the A2D ligands have an acid functionality, a further aspect of the present invention comprises a salt form containing the 2 components, particularly in a 1:1 combination. A suitable combination salt form is the salt formed by a 1:1 combination of gabapentin and sildenafil.

A suitable salt for amino acid compounds of the present invention is the hydrochloride salt. For a review on suitable salts see Stahl and Wermuth, Handbook of Pharmaceutical Salts: Properties, Selection, and Use, Wiley-VCH, Weinheim, Germany (2002).

Also within the scope of the invention are clathrates, drug-host inclusion complexes wherein, in contrast to the aforementioned solvates, the drug and host are present in non-stoichiometric amounts. For a review of such complexes, see J Pharm Sci, 64 (8), 1269-1288 by Haleblian (August 1975).

Hereinafter all references to compounds of the invention include references to salts thereof and to solvates and clathrates of compounds of the invention and salts thereof.

Also included within the present scope of the compounds of the invention are polymorphs thereof.

Prodrugs of the above compounds of the invention are included in the scope of the instant invention. The chemically modified drug, or prodrug, should have a different pharmacokinetic profile to the parent, enabling easier absorption across the mucosal epithelium, better salt formulation and/or solubility, improved systemic stability (for an increase in plasma half-life, for example). These chemical modifications may be
(1) Ester or amide derivatives which may be cleaved by, for example, esterases or lipases. For ester derivatives, the ester is derived from the carboxylic acid moiety of the drug molecule by known means. For amide derivatives, the amide may be derived from the carboxylic acid moiety or the amine moiety of the drug molecule by known means.
(2) Peptides which may be recognized by specific or nonspecific proteinases. A peptide may be coupled to the drug molecule via amide bond formation with the amine or carboxylic acid moiety of the drug molecule by known means.
(3) Derivatives that accumulate at a site of action through membrane selection of a prodrug form or modified prodrug form.
(4) Any combination of 1 to 3.

Aminoacyl-glycolic and -lactic esters are known as prodrugs of amino acids (Wermuth C. G., Chemistry and Industry, 1980:433-435). The carbonyl group of the amino acids can be esterified by known means. Prodrugs and soft drugs are known in the art (Palomino E., Drugs of the Future, 1990;15(4):361-368). The last two citations are hereby incorporated by reference.

### DETAILED DESCRIPTION OF THE INVENTION

The A2D ligands disclosed herein are prepared by methods well known to those skilled in the art. Specifically, the patents, patent applications and publications cited herein, each of which is hereby incorporated herein by reference, exemplify A2D ligands which can be used in the combinations, pharmaceutical compositions, methods and kits in accord with the present invention, and refer to methods of preparing those A2D ligands: U.S. Pat. No. 4,024,175 (specifically, gabapentin) and U.S. Pat. No. 6,028,214 (specifically, pregabalin).

The SSRI's disclosed herein are prepared by methods well known to those skilled in the art. Specifically, the following patents, patent applications and publications, each of which is hereby incorporated herein by reference, exemplify SSRI's which can be used in the combinations, pharmaceutical compositions, methods and kits of this invention, and refer to methods of preparing those SSRI's: U.S. Pat. No. 4,536,518 (specifically, sertraline); U.S. Pat. No. 4,943,590 [RE 34,712], U.S. Pat. No. 4,650,884 (specifically, citalopram); U.S. Pat. No. 3,198,834 (specifically d,l-fenfluramine); U.S. Pat. Nos. 3,912,743, 4,571,424 (specifically, femoxetine); U.S. Pat. Nos. 4,314,081, 4,626,549 (specifically, fluoxetine); U.S. Pat. No. 4,085,225 (specifically fluvoxetine); U.S. Pat. Nos. 3,912,743, 4,007,196 (specifically, paroxetine). Ifoxetine, cyanodothiepin and litoxetine are known to the skilled person and may be prepared by methods known in the art.

The SNRI's disclosed herein are prepared by methods well known to those skilled in the art. Specifically, the following patents, patent applications and publications, each of which is hereby incorporated herein by reference, exemplify SNRI's which can be used in the combinations, pharmaceutical compositions, methods and kits of this invention, and refer to methods of preparing those SNRI's: U.S. Pat. Nos. 4,229,449, 5,068,433, 5,391,735 (specifically, reboxetine); BP 908,788, 980,231, U.S. Pat. No. 3,454,554 (specifically desipramine); U.S. Pat. No. 3,399,201 (specifically, maprotiline); BP 1,177,525, U.S. Pat. No. 3,637,660 (specifically, lofepramine); U.S. Pat. No. 4,062,843 (specifically, mirtazepine); U.S. Pat. Nos. 4,314,081, 4,018,895, 4,194,009 (specifically, atomoxetine); and U.S. Pat. Nos. 3,819,706, 3,885,046 (specifically, buproprion). Oxaprotiline and fezolamine are known to the skilled person and may be prepared by methods known in the art. The inhibitors of the reuptake of both serotonin and norepinephrine disclosed herein are prepared by methods well known to those skilled in the art. Specifically, the following patents, patent applications and publications, each of which are incorporated herein by reference, exemplify compounds which inhibit both serotonin and norepinephrine uptake which can be used in the combinations, pharmaceutical compositions, methods and kits of this invention, and refer to methods of preparing those compounds: venlafaxine (Effexor®), venlafaxine metabolite O-desmethylvenlafaxine, clomipramine (Anafranil®), clomipramine metabolite desmethylclomipramine, duloxetine (Cymbalta®), milnacipran, and imipramine (Tofranil® or Janimine®).

For compounds of formula XIV of the present invention a favored embodiment is the enantiomer cis-(1S)-N-methyl-4-(3,4-dichlorophenyl)-1,2,3,4-tetrahydro-1-naphthalenamine and its pharmaceutically acceptable acid addition salts. A preferred group of the compound of formula XIV consists of the (1S)-enantiomers and the racemic mixtures of (1S)- and (1R)-enantiomers of said compounds. This group is referred to hereinafter as Group A of the present invention.

One favored group of the compounds of Group A consists of those wherein R₁ is hydrogen or methyl, R₂ is methyl and Z is selected from the group consisting of 3-chlorophenyl, 4-chlorophenyl, 3-trifluoromethylphenyl, 4-trifluoromethyl-phenyl, 3,4-dichlorophenyl, 3-bromophenyl, 4-bromophenyl, 4-methoxyphenyl and 3-trifluoromethyl-4-chloro-phenyl.

Another favored group of the compounds of Group A consists of those wherein R₁ is hydrogen or methyl, R₂ is methyl, W is hydrogen and Z is selected from the group consisting of 3,4-dichlorophenyl, 3-trifluoromethyl-phenyl, 4-chlorophenyl, 4-bromophenyl and 3-trifluoromethyl-4-chloro-phenyl.

Particularly valuable are the following compounds, in either the (1S)-enantiomeric or (1S)(1R) racemic forms, and their pharmaceutically acceptable acid addition salts:
Cis-N-methyl-4-(3,4-dichlorophenyl)-1,2,3,4-tetrahydro-1-naphthalenamine;
Cis-N-methyl-4-(4-bromophenyl)-1,2,3,4-tetrahydro-1-naphthalenamine;
Cis-N-methyl-4-(4-chlorophenyl)-1,2,3,4-tetrahydro-1-naphthalenamine;
Cis-N-methyl-4-(3-trifluoromethyl-phenyl)-1,2,3,4-tetrahydro-1-naphthalenamine;
Cis-N-methyl-4-(3-trifluoromethyl-4-chlorophenyl)-1,2,3,4-tetrahydro-1-naphthalenamine;
Cis-N,N-dimethyl-4-(4-chlorophenyl)-1,2,3,4-tetrahydro-1-naphthalenamine;
Cis-N,N-dimethyl-4-(3-trifluoromethyl-phenyl)-1,2,3,4-tetrahydro-1-naphthalenamine; and
Cis-N-methyl-4-(4-chlorophenyl)-7-chloro-1,2,3,4-tetrahydro-1-naphthalenamine.

Of interest also is the (1R)-enantiomer of cis-N-methyl-4-(3,4-dichlorophenyl)-1,2,3,4-tetrahydro-1-naphthalenamine.

Compounds of formula XIV of the present invention may be in the form of pharmaceutically acceptable salts with both organic and inorganic acids as known in the art or in the form of prodrugs or pharmaceutically acceptable salts of said prodrugs.

For compounds of formula XV of the present invention a favored embodiment is 2-[(2-ethoxyphenoxy)(phenyl)methyl]morpholine) its racemates, enantiomers and diastereoisomers. Favored embodiments of formula XV of the present invention may be in the form of pharmaceutically acceptable salts with both organic and inorganic acids or bases as known in the art or in the form of prodrugs or pharmaceutically acceptable salts of said prodrugs.

Preferred compounds of the present invention are the compounds with formula (XV) wherein n and n₁ are, independently, 1 or 2; each of the groups R and R₁ is, independently, hydrogen, methoxy, ethoxy, chlorine, trifluoromethyl or two adjacent R groups form a —O—CH₂—O— radical; R₂ is hydrogen or methyl; one of the groups R₃ and R₄ is hydrogen and the other is methyl as well as the pharmaceutically acceptable salts thereof. Particularly preferred compounds of the invention are the compounds with formula (XV) wherein n and n₁ are, independently, 1 or 2; each of the groups R and R₁ is, independently, hydrogen, methoxy, ethoxy, chlorine, trifluoromethyl or two adjacent R groups form the radical —O—CH₂—O—; R₂ and R₄, taken together, form the radical —CH₂—CH₂—, R₃ is hydrogen, methyl or isopropyl as well as the pharmaceutically acceptable salts thereof.

Examples of compounds preferred under this invention are:
2-(alpha-phenoxy-benzyl)-morpholine;
2-[alpha-(2-methoxy-phenoxy)-benzyl]-morpholine;
2-[alpha-(3-methoxy-phenoxy)-benzyl]-morpholine;
2-[alpha-(4-methoxy-phenoxy)-benzyl]-morpholine;
2-[alpha-(2-ethoxy-phenoxy)-benzyl]-morpholine;
2-[alpha-(4-chloro-phenoxy)-benzyl]-morpholine;
2-[alpha-(3,4-methylendioxy-phenoxy)-benzyl]-morpholine;
2-[alpha-(2-methoxy-phenoxy)-2-methoxy-benzyl]-morpholine;
2-[alpha-(2-ethoxy-phenoxy)-2-methoxy-benzyl]-morpholine;
2-[alpha-(2-ethoxy-phenoxy)-4-ethoxy-benzyl]-morpholine;
2-[alpha-(4-chloro-phenoxy)-4-ethoxy-benzyl]-morpholine;
2-[alpha-(2-methoxy-phenoxy)-4-ethoxy-benzyl]-morpholine;
2-[alpha-(2-methoxy-phenoxy)-2-chloro-benzyl]-morpholine;
2-[alpha-(2-ethoxy-phenoxy)-2-chloro-benzyl]-morpholine;
2-[alpha-(2-methoxy-phenoxy)-3-chloro-benzyl]-morpholine;
2-[alpha-(2-ethoxy-phenoxy)-3-chloro-benzyl]-morpholine;
2-[alpha-(2-ethoxy-phenoxy)-4-chloro-benzyl]-morpholine;
2-[alpha-(2-methoxy-phenoxy)-4-chloro-benzyl]-morpholine;
2-[alpha-(2-methoxy-phenoxy)-4-trifluoromethyl-benzyl]-morpholine;
2-[alpha-(4-ethoxy-phenoxy)-4-trifluoromethyl-benzyl]-morpholine;
2-[alpha-(2-methoxy-phenoxy)-3,4-dichloro-benzyl]-morpholine;
2-[alpha-(2-ethoxy-phenoxy)-3,4-dichloro-benzyl]-morpholine;
4-methyl-2-[alpha-(2-methoxy-phenoxy)-benzyl]-morpholine;
4-methyl-2-[alpha-(2-ethoxy-phenoxy)-benzyl]-morpholine;
4-methyl-2-[alpha-(2-methoxy-phenoxy)-3-chloro-benzyl]-morpholine;
4-methyl-2-[alpha-(2-ethoxy-phenoxy)-3-chloro-benzyl]-morpholine;
4-methyl-2-[alpha-(2-ethoxy-phenoxy)-4-chloro-benzyl]-morpholine;
4-methyl-2-[alpha-(2-methoxy-phenoxy)-4-chloro-benzyl]-morpholine;
4-methyl-2-[alpha-(2-methoxy-phenoxy)-4-trifluoromethyl-benzyl]-morpholine;
4-methyl-2-[alpha-(2-ethoxy-phenoxy)-4-trifluoromethyl-benzyl]-morpholine;
4-isopropyl-2-[alpha-(2-methoxy-phenoxy)-benzyl]-morpholine;
4-isopropyl-2-[alpha-(2-ethoxy-phenoxy)-benzyl]-morpholine;
4-isopropyl-2-[alpha-(2-methoxy-phenoxy)-3-chloro-benzyl]-morpholine;
4-isopropyl-2-[alpha-(2-ethoxy-phenoxy)-3-chloro-benzyl]-morpholine;
4-isopropyl-2-[alpha-(2-ethoxy-phenoxy)-4-chloro-benzyl]-morpholine;
4-isopropyl-2-[alpha-(2-methoxy-phenoxy)-4-chloro-benzyl]-morpholine;
4-isopropyl-2-[alpha-(2-methoxy-phenoxy)-4-trifluoromethyl-benzyl]-morpholine;
4-isopropyl-2-[alpha-(2-ethoxy-phenoxy)-4-trifluoromethyl-benzyl]-morpholine;
N-methyl-2-hydroxy-3-phenoxy-3-phenyl-propylamine;
N-methyl-2-hydroxy-3-(2-methoxy-phenoxy)-3-phenyl-propylamine;
N-methyl-2-hydroxy-3-(2-ethoxy-phenoxy)-3-phenyl-propylamine;
N-methyl-2-hydroxy-3-(4-chloro-phenoxy)-3-phenyl-propylamine;
N-methyl-2-hydroxy-3-(3,4-methylendioxy-phenoxy)-3-phenyl-propylamine;
N-methyl-2-hydroxy-3-(2-methoxy-phenoxy)-3-(2-chloro-phenyl)-propylamine;
N-methyl-2-hydroxy-3-(2-ethoxy-phenoxy)-3-(2-chloro-phenyl)-propylamine;
N-methyl-2-hydroxy-3-(2-methoxy-phenoxy)-3-(3-chloro-phenyl)-propylamine;
N-methyl-2-hydroxy-3-(2-ethoxy-phenoxy)-3-(3-chloro-phenyl)-propylamine;
N-methyl-2-hydroxy-3-(2-methoxy-phenoxy)-3-(4-chloro-phenyl)-propylamine;
N-methyl-2-hydroxy-3-(2-ethoxy-phenoxy)-3-(4-chloro-phenyl)-propylamine;
N-methyl-2-hydroxy-3-(2-methoxy-phenoxy)-3-(4-trifluoromethyl-phenyl)-propylamine;
N-methyl-2-hydroxy-3-(2-ethoxy-phenoxy)-3-(4-trifluoromethyl-phenyl)-propylamine;
N-methyl-2-hydroxy-3-(2-methoxy-phenoxy)-3-(3,4-dichloro-phenyl)-propylamine;
N-methyl-2-hydroxy-3-(2-ethoxy-phenoxy)-3-(3,4-dichloro-phenyl)-propylamine;
N-methyl-2-methoxy-3-phenoxy-3-phenyl-propylamine;
N-methyl-2-methoxy-3-(2-methoxy-phenoxy)-3-phenyl-propylamine;
N-methyl-2-methoxy-3-(2-ethoxy-phenoxy)-3-phenyl-propylamine;
N-methyl-2-methoxy-3-(4-chloro-phenoxy)-3-phenyl-propylamine;
N-methyl-2-methoxy-3-(3,4-methylenedioxy-phenoxy)-3-phenyl-propylamine;
N-methyl-2-methoxy-3-phenoxy-3-(2-chloro-phenyl)-propylamine;
N-methyl-2-methoxy-3-(2-methoxy-phenoxy)-3-(2-chloro-phenyl)-propylamine;
N-methyl-2-methoxy-3-(2-ethoxy-phenoxy)-3-(2-chloro-phenyl)-propylamine;
N-methyl-2-methoxy-3-(2-methoxy-phenoxy)-3-(3-chloro-phenyl)-propylamine;
N-methyl-2-methoxy-3-(2-ethoxy-phenoxy)-3-(3-chloro-phenyl)-propylamine;
N-methyl-2-methoxy-3-(2-methoxy-phenoxy)-3-(4-chloro-phenyl)-propylamine;
N-methyl-2-methoxy-3-(2-ethoxy-phenoxy)-3-(4-chloro-phenyl)-propylamine;
N-methyl-2-methoxy-3-(2-methoxy-phenoxy)-3-(4-trifluoromethyl-phenyl)-propylamine;
N-methyl-2-methoxy-3-(2-ethoxy-phenoxy)-3-(4-trifluoromethyl-phenyl)-propylamine;
N-methyl-2-methoxy-3-(2-methoxy-phenoxy)-3-(3,4-dichloro-phenyl)-propylamine;
N-methyl-2-methoxy-3-(2-ethoxy-phenoxy)-3-(3,4-dichloro-phenyl)-propylamine, as well as their pharmaceutically acceptable salts with both organic and inorganic acids as known in the art or in the form of prodrugs or pharmaceutically acceptable salts of said prodrugs.

A particularly preferred SSRI and A2D combination is sertraline and an A2D ligand selected from gabapentin, pregabalin, [(1R,5R,6S)-6-(Aminomethyl)bicyclo[3.2.0]hept-6-yl]acetic acid, 3-(1-Aminomethyl-cyclohexylmethyl)-4H-[1,2,4]oxadiazol-5-one and C-[1-(1H-Tetrazol-5-ylmethyl)-cycloheptyl]-methylamine, (3S,4S)-(1-Aminomethyl-3,4-dimethyl-cyclopentyl)-acetic acid, (1α,3α,5α)(3-amino-methyl-bicyclo[3.2.0]hept-3-yl)-acetic acid, (3S,5R)-3-Aminomethyl-5-methyl-octanoic acid, (3S,5R)-3-amino-5-methyl-heptanoic acid, (3S,5R)-3-amino-5-methyl-nonanoic acid, (3S,5R)-3-Amino-5-methyl-octanoic acid, and bicyclo[3.2.0]hept-3-yl)-acetic acid, and pharmaceutically acceptable salts or solvates thereof. Sertraline hydrochloride is a preferred salt.

As an alternative or further aspect of the present invention, there is provided a combination, particularly a synergistic combination, comprising gabapentin and/or pregabalin and sertraline or a pharmaceutically acceptable salt or solvate thereof.

As an alternative or further aspect of the present invention, there is provided a combination, particularly a synergistic combination, comprising pregabalin and sertraline or a pharmaceutically acceptable salt or solvate thereof.

As a yet further preferred aspect of the present invention, the combination is selected from:
gabapentin and sertraline;
gabapentin and fluoxetine;
gabapentin and paroxetine;
gabapentin and citalopram;
gabapentin and bupropion;
gabapentin and venlafaxine;
gabapentin and reboxetine;
pregabalin and sertraline;
pregabalin and fluoxetine;
pregabalin and paroxetine;
pregabalin and citalopram;
pregabalin and bupropion;
pregabalin and venlafaxine;
pregabalin and reboxetine;
pregabalin and (S,S)-reboxetine.
[(1R,5R,6S)-6-(Aminomethyl)bicyclo[3.2.0]hept-6-yl]acetic acid and sertraline;
[(1R,5R,6S)-6-(Aminomethyl)bicyclo[3.2.0]hept-6-yl]acetic acid and fluoxetine;
[(1R,5R,6S)-6-(Aminomethyl)bicyclo[3.2.0]hept-6-yl]acetic acid and paroxetine;
[(1R,5R,6S)-6-(Aminomethyl)bicyclo[3.2.0]hept-6-yl]acetic acid and citalopram;
[(1R,5R,6S)-6-(Aminomethyl)bicyclo[3.2.0]hept-6-yl]acetic acid and bupropion;
[(1R,5R,6S)-6-(Aminomethyl)bicyclo[3.2.0]hept-6-yl]acetic acid and venlafaxine;
(1α,3α,5α)(3-amino-methyl-bicyclo[3.2.0]hept-3-yl)-acetic acid and sertraline;
(1α,3α,5α)(3-amino-methyl-bicyclo[3.2.0]hept-3-yl)-acetic acid and fluoxetine;
(1α,3α,5α)(3-amino-methyl-bicyclo[3.2.0]hept-3-yl)-acetic acid and paroxetine;
(1α,3α,5α)(3-amino-methyl-bicyclo[3.2.0]hept-3-yl)-acetic acid and citalopram;
(1α,3α,5α)(3-amino-methyl-bicyclo[3.2.0]hept-3-yl)-acetic acid and bupropion;
(1α,3α,5α)(3-amino-methyl-bicyclo[3.2.0]hept-3-yl)-acetic acid and venlafaxine;
(3S,4S)-(1-Aminomethyl-3,4-dimethyl-cyclopentyl)-acetic acid and sertraline;
(3S,4S)-(1-Aminomethyl-3,4-dimethyl-cyclopentyl)-acetic acid and fluoxetine;
(3S,4S)-(1-Aminomethyl-3,4-dimethyl-cyclopentyl)-acetic acid and paroxetine;
(3S,4S)-(1-Aminomethyl-3,4-dimethyl-cyclopentyl)-acetic acid and citalopram;
(3S,4S)-(1-Aminomethyl-3,4-dimethyl-cyclopentyl)-acetic acid and bupropion; and
(3S,4S)-(1-Aminomethyl-3,4-dimethyl-cyclopentyl)-acetic acid and venlafaxine;
or pharmaceutically acceptable salts or solvates thereof.

The compounds of formulas I, II, II and IV of the present invention may be separately or in combination formulated with pharmaceutically acceptable carriers and excipients as known in the art and taught in U.S. Pat. No. 6,197,819, U.S. Pat. No. 4,024,175, U.S. Pat. No. 4,536,518 and U.S. Pat. No. 4,229,449, the disclosures of which are incorporated herein by reference, and administered in a wide variety of dosage forms as therein disclosed. Such dosage forms may be optionally modified as known in the art and in accord with the disclosures of U.S. Pat. No. 6,197,819, U.S. Pat. No. 4,024,175, U.S. Pat. No. 4,536,518 and U.S. Pat. No. 4,229,449 to include an effective amount of a compound corresponding to a compound selected from (a) a compound of formula I or formula II of the present invention, or mixtures thereof, combined with (b) an effective amount of a compound corresponding to formula XIV or (c) an effective amount of a compound corresponding to formula XV of the present invention, or a combination of (a), (b) and (c) thereby forming a unitary dosage form.

Although the aforesaid unitary dosage form provides convenience, according to the method of the present invention a pharmaceutical formulation comprising compounds corresponding to (a) formula I or formula II or a mixture thereof may be administered in combination with a pharmaceutical formulation (b) comprising at least one compound corresponding to formula XIV or a pharmaceutical formulation (c) comprising at least one compound corresponding to formula XV in a concurrent or a consecutive manner; or (a) may be administered with (b) and (c) in a concurrent or a consecutive manner.

Dosage levels of A2D ligands, SSRI's and SNRI's are well known in the art. Any effective amount of an A2D ligand may be administered with an effective amount of an SSRI or an SNRI or a combination of an SSRI and a SNRI in a concurrent or sequential manner by any means known in the art.

For example, dosage levels of a pharmaceutical formulation comprising a compound of formula I of the present invention are as follows: The quantity of active compound in a unit dose of preparation may be varied or adjusted from about 1 mg to about 300 mg/kg (milligram per kilogram) daily, based on an average 70 kg patient. A daily dose range of about 1 mg to about 50 mg/kg is preferred. The dosages, however, may be varied depending upon the requirement with a patient, the severity of the condition being treated, and the compound being employed. Determination of the proper dosage for particular situations is within the skill of the art.

An example of individual dosage levels of a pharmaceutical formulation comprising a compound of formula II of the present invention is about 5 to about 50 mg. parenterally and about 20 to about 200 mg. enterally.

An example of normally administered dosage levels of a pharmaceutical formulation comprising a compound of formula XIV of the present invention is about 0.3 mg. to about 10 mg. per kg. of body weight per day, although variations will necessarily occur depending upon the conditions of the subject being treated and the particular route of administration chosen.

In some instances, dosage levels below the lower limit of the aforesaid range may be more than adequate, while in other cases still larger doses may be employed, provided that such higher dose levels are first divided into several small doses for administration throughout the day.

An example of normally administered dosage levels of a pharmaceutical formulation comprising a compound of formula XV of the present invention suitable for oral administration to adult humans, is preferably about 5 to about 30 mg pro dose about 2 to about 4 times a day.

According to the method of the present invention, the combination of active ingredients comprising (a) an A2D ligand, including any of the compounds corresponding to formulas I or II or a mixture thereof, (b) an SSRI, including any of the compounds corresponding to formula XIV, or (c) an SNRI including any of the compounds corresponding to formula XV, or the combination of (a), (b) and (c), when used for the treatment of a subject, preferably a depressed subject, and most preferably a subject suffering from a combination of depression and anxiety, depression and sleep disorder or depression, anxiety and sleep disorder, or from post-traumatic stress, may be administered in separate parts comprising (a) and (b) or (c), or (a) and (b) and (c), or in a unitary dosage form comprising (a) and (b) or (a) and (c) or (a), (b) and (c). In any case, the active ingredients (a), (b) and (c) may be administered either alone or in combination with pharmaceutically acceptable carriers by any of the routes indicated in the incorporated references, and such administration can be carried out in both single and multiple dosages.

More particularly, according to the method of the present invention, the effective dosage level of said A2D ligand (a) may range from about 5% to about 100% of the effective dosage level when used without an SSRI (b) or an SNRI (c). In addition, the effective dosage level of said SSRI (b) or said SNRI (c) when used either separately in conjunction with A2D ligand (a), or together in conjunction with A2D ligand (a) may range from about 5% to about 100% of the effective dosage level when used without an A2D ligand.

In accord with procedures generally known and practiced in the art, when used in combination, the dosage level of (a) the A2D ligand, including any of the compounds corresponding to formulas I or II or a mixture thereof, (b) the SSRI, including any of the compounds corresponding to formula XIV and (c) the SNRI, including any of the compounds corresponding to formula XV may be adjusted to achieve the optimum effective dosage level.

The pharmaceutically active agents used in the methods and pharmaceutical compositions of this invention can be administered orally, parenterally, or topically, alone or in combination with pharmaceutically acceptable carriers or diluents, and such administration may be carried out in single or multiple doses. More particularly, the therapeutic agents of this invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutically-effective compounds of this invention are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a pharmaceutically active agent used in accordance with this invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

Additionally, it is also possible to administer the active agents used in accordance with the present invention topically, and this may be done by way of creams, jellies, gels, pastes, patches, ointments and the like, in accordance with standard pharmaceutical practice.

The anti-depressant and anxiolytic efficacy of the method and composition of the present invention may be assessed by the following tests using standard protocols to evaluate the efficacy of each active ingredient separately and in combination:

The rat Vogel Water-lick Vogel Conflict test is used to detect anxiolytic-like activity. For each experiment, naive rats are randomly divided into groups. All rats are water deprived for 24 hours prior to day one of testing. On day one, rats were placed into the test chambers and allowed to drink about five mLs of water from a drinking tube on a water bottle mounted on the outside of the test chamber. Immediately following the drinking session rats were returned to their home cages. Rats that do not meet the drinking criteria are excluded from further testing and thus are not used in the results. All rats are then water and food deprived for the next 24 hrs. On test Day two, each rat is given an appropriate treatment and placed into a test chamber and allowed to drink during a 10-minute session. Adjacent to the drink-tube is an optical lickometer with a photo beam detector that counts the number of licks during active drinking. Normally rats would emit approximately 1000 licks on day two. However, under test conditions, for every 10 licks, rats receive a mild shock through the drink-tube, which suppresses drinking behavior to ˜10% of normal levels. A conflict or anxiety-producing situation is inferred. Thus, anxiety is reflected by low amounts of drinking. Compounds that significantly increase suppressed drinking compared to concurrently run controls are presumed to possess anxiolytic-like properties. Standard benzodiazepine anxiolytic are active in this test.

The rat forced swim test is used to detect antidepressant-like activity. For each experiment, naive rats are randomly divided into groups. On day one a rat is put in a tank of water for 15 minutes, after which it is removed and allowed to dry-off. The tank of. water has a wire mesh wheel centered at the air/water interface. The rats initially try to escape the tank by swimming, with activity directed at the wheel. The number of wheel revolutions indicates the amount of activity. On next test day, which can be the following day or up to five days later, the rat is placed back in the tank. Normally the rat does not try to escape on day two and floats in the water. This is reflected in a relatively low number of wheel revolutions. Antidepressants increase the amount of swimming time on day two, measured by wheel revolutions. Drugs can be administered either acutely or repeatedly.

### Pharmaceutical Composition Examples

In the following Examples, the term ‘active compound’ or ‘active ingredient’ refers to a suitable combination or individual element of an A2D ligand and a SSRI, SNRI, SSRI/SNRI, or mixtures thereof and/or a pharmaceutically acceptable salt or solvate, according to the present invention.

(i) Tablet Compositions

The following compositions A and B can be prepared by wet granulation of ingredients (a) to (c) and (a) to (d) with a solution of povidone, followed by addition of the magnesium stearate and compression.

Composition A

| | | mg/tablet | mg/tablet |
|---|---|---|---|
| (a) | Active ingredient | 250 | 250 |
| (b) | Lactose B.P. | 210 | 26 |
| (c) | Sodium Starch Glycollate | 20 | 12 |
| (d) | Povidone B.P. | 15 | 9 |
| (e) | Magnesium Stearate | 5 | 3 |
| | | 500 | 300 |

Composition B

| | | mg/tablet | mg/tablet |
|---|---|---|---|
| (a) | Active ingredient | 250 | 250 |
| (b) | Lactose | 150 | 150 |
| (c) | Avicel PH 101 | 60 | 26 |
| (d) | Sodium Starch Glycollate | 20 | 12 |
| (e) | Povidone B.P. | 15 | 9 |
| (f) | Magnesium Stearate | 5 | 3 |
| | | 500 | 300 |

Composition C

| | mg/tablet |
|---|---|
| Active ingredient | 100 |
| Lactose | 200 |
| Starch | 50 |
| Povidone | 5 |
| Magnesium Stearate | 4 |
| | 359 |

The following compositions D and E can be prepared by direct compression of the admixed ingredients. The lactose used in formulation E is of the direct compression type.

Composition D

| | mg/tablet |
|---|---|
| Active ingredient | 250 |
| Magnesium Stearate | 4 |
| Pregelatinised Starch NF15 | 146 |
| | 400 |

Composition E

| | mg/tablet |
|---|---|
| Active ingredient | 250 |
| Magnesium Stearate | 5 |
| Lactose | 145 |
| Avicel | 100 |
| | 500 |

Composition F (Controlled release composition)

| | | mg/tablet |
|---|---|---|
| (a) | Active ingredient | 500 |
| (b) | Hydroxypropylmethylcellulose | 112 |
| | (Methocel K4M Premium) | |
| (c) | Lactose B.P. | 53 |
| (d) | Povidone B.P.C. | 28 |
| (e) | Magnesium Stearate | 7 |
| | | 700 |

The composition can be prepared by wet granulation of ingredients (a) to (c) with a solution of povidone, followed by addition of the magnesium stearate and compression.

Composition G (Enteric-Coated Tablet)

Enteric-coated tablets of Composition C can be prepared by coating the tablets with 25 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

Composition H (Enteric-Coated Controlled Release Tablet)

Enteric-coated tablets of Composition F can be prepared by coating the tablets with 50 mg/tablet of an enteric polymer such as cellulose acetate phthalate, polyvinylacetate phthalate, hydroxypropylmethyl- cellulose phthalate, or anionic polymers of methacrylic acid and methacrylic acid methyl ester (Eudgragit L). Except for Eudgragit L, these polymers should also include 10% (by weight of the quantity of polymer used) of a plasticizer to prevent membrane cracking during application or on storage. Suitable plasticizers include diethyl phthalate, tributyl citrate and triacetin.

(ii) Capsule Compositions

Composition A

Capsules can be prepared by admixing the ingredients of Composition D above and filling two-part hard gelatin capsules with the resulting mixture. Composition B (infra) may be prepared in a similar manner.

Composition B

| | | mg/capsule |
|---|---|---|
| (a) | Active ingredient | 250 |
| (b) | Lactose B.P. | 143 |
| (c) | Sodium Starch Glycollate | 25 |
| (d) | Magnesium Stearate | 2 |
| | | 420 |

Composition C

| | | mg/capsule |
|---|---|---|
| (a) | Active ingredient | 250 |
| (b) | Macrogol 4000 BP | 350 |
| | | 600 |

Capsules can be prepared by melting the Macrogol 4000 BP, dispersing the active ingredient in the melt and filling two-part hard gelatin capsules therewith.

Composition D

| | mg/capsule |
|---|---|
| Active ingredient | 250 |
| Lecithin | 100 |
| Arachis Oil | 100 |
| | 450 |

Capsules can be prepared by dispersing the active ingredient in the lecithin and arachis oil and filling soft, elastic gelatin capsules with the dispersion.

Composition E (Controlled release capsule)

| | | mg/capsule |
|---|---|---|
| (a) | Active ingredient | 250 |
| (b) | Microcrystalline Cellulose | 125 |
| (c) | Lactose BP | 125 |
| (d) | Ethyl Cellulose | 13 |
| | | 513 |

The controlled release capsule formulation can be prepared by extruding mixed ingredients (a) to (c) using an extruder, then spheronising and drying the extrudate. The dried pellets are coated with a release controlling membrane (d) and filled into two-part, hard gelatin capsules.

Composition F (Enteric capsule)

| | | mg/capsule |
|---|---|---|
| (a) | Active ingredient | 250 |
| (b) | Microcrystalline Cellulose | 125 |
| (c) | Lactose BP | 125 |
| (d) | Cellulose Acetate Phthalate | 50 |
| (e) | Diethyl Phthalate | 5 |
| | | 555 |

The enteric capsule composition can be prepared by extruding mixed ingredients (a) to (c) using an extruder, then spheronising and drying the extrudate. The dried pellets are coated with an enteric membrane (d) containing a plasticizer (e) and filled into two-part, hard gelatin capsules.

Composition G (Enteric-Coated Controlled Release Capsule)

Enteric capsules of Composition E can be prepared by coating the controlled-release pellets with 50 mg/capsule of an enteric polymer such as cellulose acetate phthalate, polyvinylacetate phthalate, hydroxypropylmethylcellulose phthalate, or anionic polymers of methacrylic acid and methacrylic acid methyl ester (Eudragit L). Except for Eudragit L, these polymers should also include 10% (by weight of the quantity of polymer used) or a plasticizer to prevent membrane cracking during application or on storage. Suitable plasticizers include diethyl phthalate, tributyl citrate and triacetin.

(iii) Intravenous injection composition

| | | |
|---|---|---|
| Active ingredient | 0.200 | g |
| Sterile, pyrogen-free phosphate buffer (pH 9.0) to | 10 | ml |

The active ingredient is dissolved in most of the phosphate buffer at 35-40° C, then made up to volume and filtered through a sterile micropore filter into sterile 10 ml glass vials (Type 1) which are sealed with sterile closures and overseals.

(iv) Intramuscular injection composition

| | | |
|---|---|---|
| Active ingredient | 0.20 | g |
| Benzyl Alcohol | 0.10 | g |
| Glycofurol 75 | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (Type 1).

(v) Syrup composition

| | | |
|---|---|---|
| Active ingredient | 0.25 | g |
| Sorbitol Solution | 1.50 | g |
| Glycerol | 1.00 | g |
| Sodium Benzoate | 0.005 | g |
| Flavor | 0.0125 | ml |
| Purified Water q.s. to | 5.0 | ml |

The sodium benzoate is dissolved in a portion of the purified water and the sorbitol solution added. The active ingredient is added and dissolved. The resulting solution is mixed with the glycerol and then made up to the required volume with the purified water.

(vi) Suppository composition

| | mg/suppository |
|---|---|
| Active ingredient | 250 |
| Hard Fat, BP (Witepsol H15 - Dynamit NoBel) | 1770 |
| | 2020 |

One-fifth of the Witepsol H15 is melted in a steam-jacketed pan at 45° C. maximum. The active ingredient is sifted through a 200 lm sieve and added to the molten base with mixing, using a Silverson fitted with a cutting head, until a smooth dispersion is achieved. Maintaining the mixture at 45° C., the remaining Witepsol H15 is added to the suspension which is stirred to ensure a homogenous mix. The entire suspension is then passed through a 250 lm stainless steel screen and, with continuous stirring, allowed to cool to 40° C. At a temperature of 38-40° C., 2.02 g aliquots of the mixture are filled into suitable plastic moulds and the suppositories allowed to cool to room temperature.

(vii) Pessary composition

| | mg/pessary |
|---|---|
| Active ingredient (631 m) | 250 |
| Anhydrous Dextrose | 380 |
| Potato Starch | 363 |
| Magnesium Stearate | 7 |
| | 1000 |

The above ingredients are mixed directly and pessaries prepared by compression of the resulting mixture.

(viii) Transdermal composition

| | | |
|---|---|---|
| Active ingredient | 200 | mg |
| Alcohol USP | 0.1 | ml |
| Hydroxyethyl cellulose | | |

The active ingredient and alcohol USP are gelled with hydroxyethyl cellulose and packed in a transdermal device with a surface area of 10 cm².

1. A method of treating depression and/or anxiety in a mammal, including a human, comprising: administering to said mammal a combination of active agents comprising;
(a) an alpha-2-delta (A2D) ligand or a prodrug thereof, or a pharmaceutically acceptable salt of said A2D ligand or said prodrug and, active agents selected from;
(b) a selective serotonin re-uptake inhibitor (SSRI) or a prodrug thereof or a pharmaceutically acceptable salt of said SSRI or said prodrug,
(c) a selective noradrenaline re-uptake inhibitor (SNRI) or a prodrug thereof or a pharmaceutically acceptable salt of said SNRI or said prodrug and mixtures of (b) and (c),
wherein said active agents (a), (b) and (c) above are administered in amounts that are effective in said combination.

2. The method according to claim 1, wherein (b) and (c) are the same active agent.

3. The method according to claim 1, wherein said depression and/or anxiety is accompanied with at least one other concomitant disease, disorder or condition.

4. The method according to claim 1, wherein said active agents (a) and (b), (a) and (c), or (a), (b), and (c) are administered concurrently or consecutively.

5. The method according to claim 1 wherein said A2D ligand is selected from the group consisting of gabapentin, pregabalin, or a prodrug thereof or a pharmaceutically acceptable salt of said A2D ligand or said prodrug.

6. The method according to claim 1 wherein said SSRI is selected from the group consisting of: sertraline, fluoxetine, fluvoxamine, paroxetine, citalopram, d,l-fenfluramine, femoxetine, ifoxetine, cyanodothiepin, litoxetine, cericlamine, dapoxetine, nefazaodone, and trazodone, or a prodrug thereof or a pharmaceutically acceptable salt of said SSRI or said prodrug.

7. The method according to claim 1 wherein said SNRI is selected from the group consisting of: reboxetine, desipramine, maprotiline, lofepramine, mirtazepine, oxaprotiline, fezolamine, atomoxetine, buproprion, mianserin, or a prodrug thereof or a pharmaceutically acceptable salt of said SNRI or said prodrug.

8. The method according to claim 3 of treating depression with concomitant anxiety.

9. The method according to claim 3 of treating post-traumatic stress disorder.

10. The method according to claim 3 of treating depression with concomitant sleep disorders including insomnia.

11. The method according to claim 1 of treating depression with concomitant anxiety and sleep disorders including insomnia.

12. The method according to claim 1 of treating attention deficit hyperactivity disorder (ADHD) with concomitant anxiety.

13. The method according to claim 1 of treating anxiety with concomitant sleep disorders including insomnia.

14. The method according to claim 1 wherein
(a) comprises (i) a compound having the formula 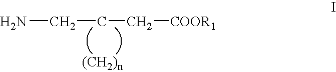
wherein with regard to formula II, R₁ is a hydrogen atom or a lower alkyl and n is 4, 5, or 6 wherein the lower alkyls are straight or branched chain alkyls containing up to 8, and preferably up to 4 carbon atoms selected from methyl, ethyl, isopropyl, and tert.-butyl, or a prodrug thereof, or a pharmaceutically acceptable salt thereof or said prodrug; or
(ii) a compound having the formula 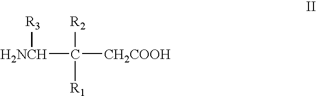
wherein with regard to formula I, R₁ is a straight or branched alkyl of from 1 to 6 carbons, phenyl or cycloalkyl having from 3 to 6 carbon atoms; R₂ is hydrogen or methyl; and R₃ is hydrogen, methyl, or carboxyl; said formula including the racemates or the individual enantiomers thereof; or a prodrug thereof, or a pharmaceutically acceptable salt thereof or said prodrug; or a mixture of said compound of formula I with said compound of formula II, or said prodrugs, pharmaceutically acceptable salts or salts of said prodrugs corresponding to said compounds of formula I and formula II.

15. The method according to claim 1 wherein
(b) comprises an effective amount of a compound selected from the group consisting of cis-isomeric bases of the formula 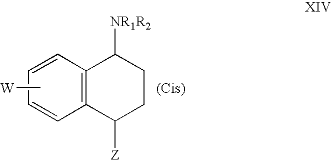
wherein with regard to formula XIV
R₁ is selected from the group consisting of hydrogen and methyl,
R₂ is methyl,
Z is selected from the group consisting of 3-chlorophenyl, 4-chlorophenyl, 4-methoxyphenyl, 3-trifluoromethyl-phenyl, 4-trifluoromethyl-phenyl, 3,4-dichlorophenyl, 3-bromophenyl, 4-bromophenyl and 3-trifluoromethyl-4-chloro-phenyl, and
W is selected from the group consisting of hydrogen, fluoro, chloro, bromo, trifluoromethyl and alkoxy of from 1 to 3 carbon atoms, with said compound being either the (1S)-enantiomer or the racemic mixture of the (1S)-enantiomer with the corresponding (1R)-enantiomer or a prodrug thereof or a pharmaceutically acceptable salt thereof or of said prodrug.

16. The method according to claim 1 wherein
(c) comprises an effective amount of a compound having the formula 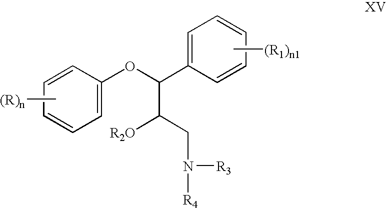
wherein with regard to formula XV
n and n₁ are independently 1, 2 or 3;
each of the groups R and R₁ which may be the same or different is hydrogen;
halogen; halo-C₁-C₆-alkyl; hydroxy; C₁-C₆ alkoxy; C₁-C₆ alkyl unsubstituted or substituted by one or more hydroxy or C₁-C₆ alkoxy groups; phenyl-C₁-C₆-alkyl or phenyl-C₁-C₆-alkoxy in which the phenyl group may be unsubstituted or substituted by one or more substituents chosen from the group consisting of C₁-C₆ alkyl, halogen, C₁-C₆-alkoxy, hydroxy and halo-C₁-C₆ alkyl;
R₃ is hydrogen, C₁-C₆ alkyl unsubstituted or substituted by one or more halogen, hydroxy or C₁-C₆ alkoxy groups C₂-C₄ alkenyl; C₂-C₄ alkynyl; phenyl-C₁-C₄-alkyl in which the phenyl group may be unsubstituted or substituted by one or more C₁-C₆ alkyl, halogen, halo-C₁-C₆ alkyl, hydroxy and C₁-C₆ alkoxy groups; or C₃-C₇ cycloalkyl unsubstituted or substituted by one or more C₁-C₆ alkyl, halogen, halo-C₁-C₆ alkyl, hydroxy and C₁-C₆ alkoxy groups;
R₂ and R₄, taken together, form the radical —CH₂—CH₂—, with said compound of formula IV being either a racemic mixture or individual enantiomeric isomers and diastereoisomers or mixtures thereof, or a prodrug thereof, or a pharmaceutically acceptable salt thereof or of said prodrug.

17. A method according to claim 1 wherein
(c) comprises an effective amount of a compound having the formula 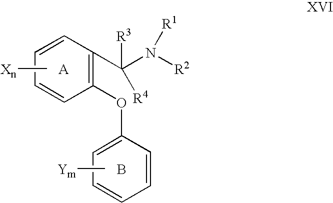
wherein phenyl ring A and phenyl ring B can each, independently, be replaced by a naphthyl group, and wherein when phenyl ring A is replaced by a naphthyl group, the ethereal oxygen of structure XVI and the carbon to which R³, R⁴and NR¹R² are attached, are attached to adjacent ring carbon atoms of the naphthyl group and neither of said adjacent ring carbon atoms is also adjacent to a fused ring carbon atom of said naphthyl group;
n and m are, selected, independently, from one, two and three;
R¹ and R² are selected, independently, from hydrogen, (C₁-C₄)alkyl, (C₂-C₄)alkenyl, and (C₂-C₄)alkynyl, or R¹ and R², together with the nitrogen to which they are attached, form a four to eight membered saturated ring containing one or two heteroatoms, including the nitrogen to which R¹ and R² are attached, wherein the second heteroatom, when present, is selected from oxygen, nitrogen and sulfur, with the proviso that said ring can not contain two adjacent oxygen atoms or two adjacent sulfur atoms, and wherein said ring may optionally be substituted at available binding sites with from one to three substituents selected, independently, from hydroxy and (C₁-C₆)alkyl;
R³ and R⁴ are selected, independently, from hydrogen and (C₁-C₄) alkyl optionally substituted with from one to three fluorine atoms, or R³ and R⁴, together with the carbon to which they are attached, form a four to eight membered saturated carbocyclic ring, and wherein said ring may optionally be substituted at available binding sites with from one to three substituents selected, independently, from hydroxy and (C₁-C₆)alkyl; or R² and R³, together with the nitrogen to which R is attached and the carbon to which R³ is attached, form a four to eight membered saturated ring containing one or two heteroatoms, including the nitrogen to which R² is attached, wherein the second heteroatom, when present, is selected from oxygen, nitrogen and sulfur, with the proviso that said ring can not contain two adjacent oxygen atoms or two adjacent sulfur atoms, and wherein said ring may optionally be substituted at available binding sites with from one to three substituents selected, independently, from hydroxy and (C₁-C₆)alkyl;
each X is selected, independently, from hydrogen, halo, (C₁-C₄)alkyl optionally substituted with from one to three fluorine atoms, (C₁-C₄)alkoxy optionally substituted with from one to three fluorine atoms, cyano, nitro, amino, (C₁-C₄)alkylamino, di-[(C₁-C₄)alkyl]amino, NR⁵(C═O)(C₁-C₄)alkyl, SO₂NR⁵R⁶ and SOₚ(C₁-C₆)alkyl, wherein R⁵ and R⁶ are selected, independently, from hydrogen and (C₁-C₆)alkyl, and p is zero, one or two; and
each Y is selected, independently, from hydrogen, (C₁-C₆)alkyl and halo; with the proviso that: (a) no more than one of NR¹R², CR³R⁴ and R²NCR³ can form a ring; and (b) at least one X must be other than hydrogen when (i) R³ and R⁴ are both hydrogen, (ii) R¹ and R² are selected, independently, from hydrogen and (C₁-C₄)alkyl, and (iii) ring B is mono- or disubstituted with, respectively, one or two halo groups;
or a pharmaceutically acceptable salt thereof.

18. The method according to claim 17, wherein said compound or salt is selected from the following compounds and their pharmaceutically acceptable salts:
[2-(3,4-Dichlorophenoxy)-5-fluorobenzyl]-dimethylamine;
[2-(3,4-Dichlorophenoxy)-5-fluorobenzyl]-methylamine;
[2-(3,4-Dichlorophenoxy)-5-trifluoromethylbenzyl]-dimethylamine;
N-[4-(3,4-Dichlorophenoxy)-3-dimethylaminomethylphenyl]-acetamide;
{1-[2-(3,4-Dichlorophenoxy)phenyl]-ethyl }-dimethylamine;
[2-(3,4-Dichlorophenoxy)-4-trifluoromethylbenzyl]-dimethylamine;
[2-(3,4-Dichlorophenoxy)-4-trifluoromethylbenzyl]-methylamine;
[4-Chloro-2-(3,4-dichlorophenoxy)-benzyl]-methylamine;
{1-[2-(3,4-Dichlorophenoxy)-5-fluorophenyl]-ethyl}-methylamine;
{1-[2-(3,4-Dichlorophenoxy)phenyl}-ethyl}-methylamine;
{1-[2-(4-Chlorophenoxy)phenyl]ethyl}-methylamine;
[2-(3,4-Dichlorophenoxy)-5-methoxybenzyl]-methylamine;
[2-(4-Chlorophenoxy)-5-fluorobenzyl]-methyl amine;
{1-[2-(4-Chlorophenoxy)-5-fluorophenyl]-ethyl}-methylamine;
[2-(3,4-Dichlorophenoxy)-5-methylbenzyl]-dimethylamine;
[4-Bromo-2-(3,4-dichlorophenoxy)-benzyl]-methylamine;
[5-Bromo-2-(3,4-dichlorophenoxy)-benzyl]-methylamine;
[2-(3,4-Dichlorophenoxy)-4,5-dimethoxybenzyl]-methylamine;
[2-(3,4-Dichlorophenoxy)-4-methoxybenzyl]-dimethylamine;
4-(3,4-Dichlorophenoxy)-3-methylaminomethyl-benzonitrile;
[2-(3,4-Dichlorophenoxy)-4,5-dimethylbenzyl]-methylamine;
3-(3,4-Dichlorphenoxy)-4-methylaminomethyl-benzonitrile;
(+)-{1-[2-(3,4-Dichlorophenoxy)-5-fluorophenyl]-ethyl}-methylamine;
(−)-{1-[2-(3,4-Dichlorophenoxy)-5-fluorophenyl]-ethyl }-methylamine;
[2-(3,4-Dichlorophenoxy)-5-trifluoromethyl-benzyl]-methyl amine;
[2-(3,4-Dichlorophenoxy)-4-methoxybenzyl]-methylamine;
[2-(4-Chloro-3-fluorophenoxy)-5-fluorobenzyl]-methylamine;
[2-(3-Chloro-4-fluorophenoxy)-5-fluorobenzyl]-methylamine;
(±)-2-[2-(3,4-Dichlorophenoxy)-5-fluorophenyl]-pyrrolidine;
(−)-2-[2-(3,4-Dichlorophenoxy)-5-fluorophenyl]-pyrrolidine;
(+)-2-[2-(3,4-Dichlorophenoxy)-5-fluorophenyl]-pyrrolidine; and
2-[2-(3,4-Dichlorophenoxy)-5-fluorophenyl]-N-methylpyrrolidine.

19. The method according to claim 14 wherein (b) comprises an effective amount of the compound of formula XIV, wherein, with regard to formula XIV, R₁, R₂, W and Z are as defined in claim 14 and (c) comprises an effective amount of the compound of formula XV, wherein, with regard to formula XV, n, n₁, R, R₁, R₂, R₃ and R₄ are as defined in claim 15.

20. The method according to claim 19 wherein the compound of formula I corresponds to S-(+)-4-amino-3-(2-methylpropyl) butanoic acid, and the compound of formula II corresponds to 1-(aminomethyl)cyclohexanacetic acid, and the compound of formula XIV corresponds to (1S-cis)-4-(3,4-dichlorophenyl)-1,2,3,4-tetrahydro-N-methyl-1-nanphthalenamine, and the compound of formula IV corresponds to (RS)-2-[(RS-alpha (2-ethoxyphenoxy)benzyl]-morpholine.

21. The method according to claim 1 of treating a mammal, including a human, for depression and depression with at least one concomitant disease, disorder or condition, selected from the group consisting of; anxiety, post traumatic stress disorder and sleep disorders including insomnia.

22. A method according to claim 1 of treating depression or anxiety with one or more concomitant disease, disorder or condition selected from the group consisting of: anxiety, post traumatic stress disorder, panic phobias, obsessive compulsive disorder (OCD), borderline personality disorder, sleep disorder, psychosis, seizures, dyskinesia, symptoms of Huntington's or Parkinson's diseases, spasticity, seizures resulting from epilepsy, cerebral ischemia, anorexia, faintness attacks, hypokinesia, cranial traumas, deteriorated cerebral function in geriatric patients, chemical dependencies, premature ejaculation, post myocardial infarction, regulation of immune response, immune system disorders, premenstrual syndrome (PMS) associated mood and appetite disorder, hot flashes, cancer, potential stenosis, modification of feeding behavior, carbohydrate cravings, late luteal phase dysphoric disorder, attention deficit hyperactivity disorder (ADHD), and tobacco withdrawal-associated symptoms.

23. The method according to claim 1 of treating circadian rhythm disorders, psychoactive substance abuse and dependence, paraphilias, sexual dysfunctions, stress related illnesses and personality disorders manifested by anger, rejection sensitivity, low mental or physical energy, circadian rhythm disorders, personality disorders including borderline and antisocial personality disorders, hyopochondriasis, psychoactive substance use disorders, sexual disorders, schizophrenia, and related symptoms including stress, worry, and lack of mental or physical energy.

24. The method according to claim 19 of treating a mammal, including a human, for depression and depression with at least one concomitant disease, disorder or condition, selected from the group consisting of;
anxiety, post traumatic stress disorder and sleep disorders including insomnia.

25. A method according to claim 19 of treating depression with one or more concomitant disease, disorder or condition selected from the group consisting of: anxiety, post traumatic stress disorder, panic phobias, obsessive compulsive disorder (OCD), borderline personality disorder, sleep disorder, psychosis, seizures, dyskinesia, symptoms of Huntington's or Parkinson's diseases, spasticity, seizures resulting from epilepsy, cerebral ischemia, anorexia, faintness attacks, hypokinesia, cranial traumas, deteriorated cerebral function in geriatric patients, chemical dependencies, premature ejaculation, post myocardial infarction, regulation of immune response, immune system disorders, premenstrual syndrome (PMS) associated mood and appetite disorder, hot flashes, cancer, potential stenosis, modification of feeding behavior, carbohydrate cravings, late luteal phase dysphoric disorder, attention deficit hyperactivity disorder (ADHD), and tobacco withdrawal-associated symptoms.

26. The method according to claim 19 of treating circadian rhythm disorders, psychoactive substance abuse and dependence, paraphilias, sexual dysfunctions, stress related illnesses and personality disorders manifested by anger, rejection sensitivity, low mental or physical energy, circadian rhythm disorders, personality disorders including borderline and antisocial personality disorders, hyopochondriasis, psychoactive substance use disorders, sexual disorders, schizophrenia, and related symptoms including stress, worry, and lack of mental or physical energy.

27. The method according to claim 1, wherein the A2D ligand is pregabalin and the SSRI is sertraline.

28. A pharmaceutical composition comprising a therapeutically effective amount of active agents comprising;
(a) an A2D ligand or a prodrug thereof, or a pharmaceutically acceptable salt of said A2D ligand or said prodrug and active agents selected from;
(b) a selective serotonin re-uptake inhibitor (SSRI) or a prodrug thereof or a pharmaceutically acceptable salt of said SSRI or said prodrug,
(c) a selective noradrenaline re-uptake inhibitor (SNRI) or a prodrug thereof or a pharmaceutically acceptable salt of said SNRI or said prodrug and mixtures of (b) and (c).

29. The pharmaceutical composition of claim 28, wherein (b) and (c) are the same active agent.

30. The pharmaceutical composition of claim 28, additionally comprising a pharmaceutically acceptable vehicle, carrier or diluent.

31. The pharmaceutical composition of claim 30 wherein said A2D ligand is selected from the group consisting of gabapentin, pregabalin or a prodrug thereof or a pharmaceutically acceptable salt of said A2D ligand or said prodrug.

32. The pharmaceutical composition of claim 31 wherein said SSRI is selected from sertraline, fluoxetine, fluvoxamine, paroxetine, citalopram, d,l-fenfluramine, femoxetine, ifoxetine, cyanodothiepin, litoxetine, cericlamine, dapoxetine, nefazaodone, trazodone, a prodrug thereof or a pharmaceutically acceptable salt of said SSRI or said prodrug.

33. The pharmaceutical composition of claim 32 wherein said SNRI is selected from reboxetine, desipramine, maprotiline, lofepramine, mirtazepine, oxaprotiline, fezolamine, atomoxetine and buproprion, mianserin, a prodrug thereof or a pharmaceutically acceptable salt of said SNRI or said prodrug.

34. A pharmaceutical composition according to claim 28, wherein the A2D ligand is pregabalin and the SSRI is sertraline.

35. A pharmaceutical composition of claim 29 wherein (a) comprises
(i) a compound having the formula 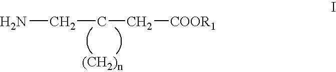
wherein with regard to formula II, R₁ is a hydrogen atom or a lower alkyl and n is 4, 5, or 6 wherein the lower alkyls are straight or branched chain alkyls containing up to 8, and preferably up to 4 carbon atoms selected from methyl, ethyl, isopropyl, and tert.-butyl, or a prodrug thereof, or a pharmaceutically acceptable salt thereof or said prodrug; or,
(ii) a compound having the formula 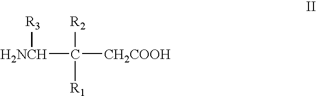
wherein with regard to formula I, R₁ is a straight or branched alkyl of from 1 to 6 carbons, phenyl or cycloalkyl having from 3 to 6 carbon atoms; R₂ is hydrogen or methyl; and R₃ is hydrogen, methyl, or carboxyl; said formula including the racemates or the individual enantiomers thereof; or a prodrug thereof, or a pharmaceutically acceptable salt thereof or said prodrug; or a mixture of said compound of formula I with said compound of formula II, or said prodrugs, pharmaceutically acceptable salts or salts of said prodrugs corresponding to said compounds of formula I and formula II.

36. A pharmaceutical composition of claim 35 wherein,
(b) comprises an effective amount of a compound selected from the group consisting of cis-isomeric bases of the formula 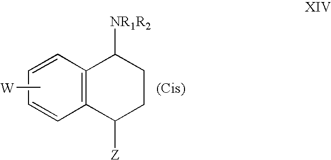
wherein with regard to formula XIV
R₁ is selected from the group consisting of hydrogen and methyl,
R₂ is methyl,
Z is selected from the group consisting of 3-chlorophenyl, 4-chlorophenyl, 4-methoxyphenyl, 3-trifluoromethyl-phenyl, 4-trifluoromethyl-phenyl, 3,4-dichlorophenyl, 3-bromophenyl, 4-bromophenyl and 3-trifluoromethyl-4-chloro-phenyl, and
W is selected from the group consisting of hydrogen, fluoro, chloro, bromo, trifluoromethyl and alkoxy of from 1 to 3 carbon atoms, with said compound being either the (1S)-enantiomer or the racemic mixture of the (1S)-enantiomer with the corresponding (1R)-enantiomer or a prodrug thereof or a pharmaceutically acceptable salt thereof or of said prodrug.

37. A pharmaceutical composition of claim 36 wherein,
(c) comprises an effective amount of a compound having the formula 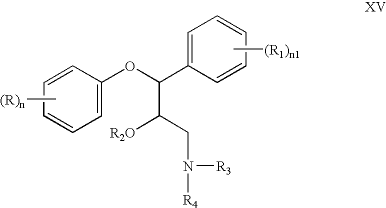
wherein with regard to formula XV
n and n₁ are independently 1, 2 or 3;
each of the groups R and R₁ which may be the same or different is hydrogen;
halogen; halo-C₁-C₆-alkyl; hydroxy; C₁-C₆ alkoxy; C₁-C₆ alkyl unsubstituted or substituted by one or more hydroxy or C₁-C₆ alkoxy groups; phenyl-C₁-C₆-alkyl or phenyl-C₁-C₆-alkoxy in which the phenyl group may be unsubstituted or substituted by one or more substituents chosen from the group consisting of C₁-C₆ alkyl, halogen, C₁-C₆-alkoxy, hydroxy and halo-C₁-C₆ alkyl;
R₃ is hydrogen, C₁-C₆ alkyl unsubstituted or substituted by one or more halogen, hydroxy or C₁-C₆ alkoxy groups C₂-C₄ alkenyl; C₂-C₄ alkynyl; phenyl-C₁-C₄-alkyl in which the phenyl group may be unsubstituted or substituted by one or more C₁-C₆ alkyl, halogen, halo-C₁-C₆ alkyl, hydroxy and C₁-C₆ alkoxy groups; or C₃-C₇ cycloalkyl unsubstituted or substituted by one or more C₁-C₆ alkyl, halogen, halo-C₁-C₆ alkyl, hydroxy and C₁-C₆ alkoxy groups;
R₂ and R₄, taken together, form the radical —CH₂—CH₂—, with said compound of formula XV being either a racemic mixture or individual enantiomeric isomers and diastereoisomers or mixtures thereof, or a prodrug thereof, or a pharmaceutically acceptable salt thereof or of said prodrug.

38. A pharmaceutical composition of claim 35 wherein,
(c) comprises an effective amount of a compound having the formula 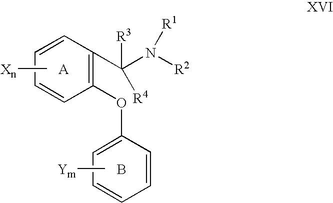
wherein phenyl ring A and phenyl ring B can each, independently, be replaced by a naphthyl group, and wherein when phenyl ring A is replaced by a naphthyl group, the ethereal oxygen of structure XVI and the carbon to which R³, R⁴ and NR¹R² are attached, are attached to adjacent ring carbon atoms of the naphthyl group and neither of said adjacent ring carbon atoms is also adjacent to a fused ring carbon atom of said naphthyl group;
n and m are, selected, independently, from one, two and three;
R¹ and R² are selected, independently, from hydrogen, (C₁-C₄)alkyl, (C₂-C₄)alkenyl, and (C₂-C₄)alkynyl, or R¹ and R², together with the nitrogen to which they are attached, form a four to eight membered saturated ring containing one or two heteroatoms, including the nitrogen to which R¹ and R² are attached, wherein the second heteroatom, when present, is selected from oxygen, nitrogen and sulfur, with the proviso that said ring can not contain two adjacent oxygen atoms or two adjacent sulfur atoms, and wherein said ring may optionally be substituted at available binding sites with from one to three substituents selected, independently, from hydroxy and (C₁-C₆)alkyl;
R³ and R⁴ are selected, independently, from hydrogen and (C₁-C₄) alkyl optionally substituted with from one to three fluorine atoms, or R³ and R⁴, together with the carbon to which they are attached, form a four to eight membered saturated carbocyclic ring, and wherein said ring may optionally be substituted at available binding sites with from one to three substituents selected, independently, from hydroxy and (C₁-C₆)alkyl; or R² and R³, together with the nitrogen to which R² is attached and the carbon to which R³ is attached, form a four to eight membered saturated ring containing one or two heteroatoms, including the nitrogen to which R² is attached, wherein the second heteroatom, when present, is selected from oxygen, nitrogen and sulfur, with the proviso that said ring can not contain two adjacent oxygen atoms or two adjacent sulfur atoms, and wherein said ring may optionally be substituted at available binding sites with from one to three substituents selected, independently, from hydroxy and (C₁-C₆)alkyl;
each X is selected, independently, from hydrogen, halo, (C₁-C₄)alkyl optionally substituted with from one to three fluorine atoms, (C₁-C₄)alkoxy optionally substituted with from one to three fluorine atoms, cyano, nitro, amino, (C₁-C₄)alkylamino, di-[(C₁-C₄)alkyl]amino, NR⁵(C═O)(C₁-C₄)alkyl, SO₂NR⁵R⁶ and SOₚ(C₁-C₆)alkyl, wherein R⁵ and R⁶ are selected, independently, from hydrogen and (C₁-C₆)alkyl, and p is zero, one or two; and
each Y is selected, independently, from hydrogen, (C₁-C₆)alkyl and halo; with the proviso that: (a) no more than one of NR¹R², CR³R⁴ and R NCR can form a ring; and (b) at least one X must be other than hydrogen when (i) R³ and R⁴ are both hydrogen, (ii) R¹ and R² are selected, independently, from hydrogen and (C₁-C₄)alkyl, and (iii) ring B is mono- or disubstituted with, respectively, one or two halo groups;
or a pharmaceutically acceptable salt thereof.

39. A pharmaceutical composition according to claim 38, wherein said compound or salt is selected from the following compounds and their pharmaceutically acceptable salts:
[2-(3,4-Dichlorophenoxy)-5-fluorobenzyl]-dimethylamine;
[2-(3,4-Dichlorophenoxy)-5-fluorobenzyl]-methylamine;
[2-(3,4-Dichlorophenoxy)-5-trifluoromethylbenzyl]-dimethylamine;
N-[4-(3,4-Dichlorophenoxy)-3-dimethylaminomethylphenyl]-acetamide;
1-[2-(3,4-Dichlorophenoxy)phenyl]-ethyl}-dimethylamine;
[2-(3,4-Dichlorophenoxy)-4-trifluoromethylbenzyl]-dimethylamine;
[2-(3,4-Dichlorophenoxy)-4-trifluoromethylbenzyl]-methylamine;
[4-Chloro-2-(3,4-dichlorophenoxy)-benzyl]-methylamine;
{1-[2-(3,4-Dichlorophenoxy)-5-fluorophenyl]-ethyl}-methylamine;
{1-[2-(3,4-Dichlorophenoxy)phenyl}-ethyl}-methylamine;
{1-[2-(4-Chlorophenoxy)phenyl]ethyl}-methylamine;
[2-(3,4-Dichlorophenoxy)-5-methoxybenzyl]-methylamine;
[2-(4-Chlorophenoxy)-5-fluorobenzyl]-methylamine;
{1-[2-(4-Chlorophenoxy)-5-fluorophenyl]-ethyl}-methylamine;
[2-(3,4-Dichlorophenoxy)-5-methylbenzyl]-dimethylamine;
[4-Bromo-2-(3,4-dichlorophenoxy)-benzyl]-methylamine;
[5-Bromo-2-(3,4-dichlorophenoxy)-benzyl]-methylamine;
[2-(3,4-Dichlorophenoxy)-4,5-dimethoxybenzyl]-methylamine;
[2-(3,4-Dichlorophenoxy)-4-methoxybenzyl]-dimethylamine;
4-(3,4-Dichlorophenoxy)-3-methylaminomethyl-benzonitrile;
[2-(3,4-Dichlorophenoxy)-4,5-dimethylbenzyl]-methylamine;
3-(3,4-Dichlorphenoxy)-4-methylaminomethyl-benzonitrile;
(+)-{1-[2-(3,4-Dichlorophenoxy)-5-fluorophenyl]-ethyl }-methylamine;
(−)-{1-[2-(3,4-Dichlorophenoxy)-5-fluorophenyl]-ethyl }-methylamine;
[2-(3,4-Dichlorophenoxy)-5-trifluoromethyl-benzyl]-methylamine;
[2-(3,4-Dichlorophenoxy)-4-methoxybenzyl]-methyl amine;
[2-(4-Chloro-3-fluorophenoxy)-5-fluorobenzyl]-methylamine;
[2-(3-Chloro-4-fluorophenoxy)-5-fluorobenzyl]-methylamine;
(±)-2-[2-(3,4-Dichlorophenoxy)-5-fluorophenyl]-pyrrolidine;
(−)-2-[2-(3,4-Dichlorophenoxy)-5-fluorophenyl]-pyrrolidine;
(+)-2-[2-(3,4-Dichlorophenoxy)-5-fluorophenyl]-pyrrolidine; and
2-[2-(3,4-Dichlorophenoxy)-5-fluorophenyl]-N-methylpyrrolidine.

40. A pharmaceutical composition of claim 37 wherein the compound of formula I corresponds to 1-(aminomethyl)cyclohexanacetic acid and the compound of Formula II corresponds to S-(+)-4-amino-3-(2-methylpropyl) butanoic acid, and the compound of formula XIV corresponds to (1S-cis)-4-(3,4-dichlorophenyl)-1,2,3,4-tetrahydro-N-methyl-1-nanphthalenamine and the compound of formula XV corresponds to (RS)-2-[(RS-alpha (2-ethoxyphenoxy)benzyl]-morpholine or individual enantiomeric isomers and diastereoisomers or mixtures thereof, or a prodrug thereof, or a pharmaceutically acceptable salt thereof or of said prodrug.
